SEQUENCE LISTING (37 sequences)

SEQ ID NO: 1 - protein, 13 aa, Homo sapiens
CATWDRRDYKKLF

SEQ ID NO: 2 - protein, 16 aa, Homo sapiens
CALGVLPTVTGGGLIF

SEQ ID NO: 3 - protein, 6 aa, Homo sapiens
VTNTFY

SEQ ID NO: 4 - protein, 8 aa, Homo sapiens
YDVSTARD

SEQ ID NO: 5 - protein, 7 aa, Homo sapiens
TSWWSYY

SEQ ID NO: 6 - protein, 3 aa, Homo sapiens
QGS

SEQ ID NO: 7 - protein, 226 aa, Homo sapiens
SSNLEGRTKSVTRQTGSSAEITCDLTVTNTFYIHWYLHQEGKAPQRLLYYDVSTARDVLESGLSPGKYYTHTPRRWSWILRLQNLIENDSGVYYCATWDRRDYKKLFGSGTTLVVTDKQLDADVSPKPTIFLPSIAETKLQKAGTYLCLLEKFFPDVIKIHWQEKKSNTILGSQEGNTMKTNDTYMKFSWLTVPEKSLDKEHRCIVRHENNKNGVDQEIIFPPIKT

SEQ ID NO: 8 - protein, 209 aa, Artificial Sequence
  The sequence of the extracellular region less the connecting peptide region of the delta chain of a mutant TCR
AQKVTQAQSSVSMPVRKAVTLNCLYETSWWSYYIFWYKQLPSKEMIFLIRQGSDEQNAKSGRYSVNFKKAAKSVALTISALQLEDSAKYFCALGVLPTVTGGGLIFGKGTRVTVEPNSQPHTKPSVFVMKNGTNVACLVKEFYPKDIRINLVSSKKITEFDPAIVISPSGKYNAVKLGKYEDSNSVTCSVQHDNKTVHSTDFEVKTDST

SEQ ID NO: 9 - protein, 12 aa, Homo sapiens
CALWEVDYKKLF

SEQ ID NO: 10 - protein, 16 aa, Homo sapiens
CALGEPVLFAVRGLIF

SEQ ID NO: 11 - protein, 16 aa, Homo sapiens
CACDLLGDRYTDKLIF

SEQ ID NO: 12 - DNA, 104 bp, Artificial Sequence
  GeCKO gRNA sequence
gaattactctcacttccaccacccgatgtatggaaactgctatactttcaatgacaagaacaactccaacctctggatgtcttccatgcctggaatcaacaacg

SEQ ID NO: 13 - DNA, 104 bp, Artificial Sequence; identical to SEQ ID NO: 12
  gRNA sequence

SEQ ID NO: 14 - protein, 209 aa, Homo sapiens
AQKVTQAQSSVSMPVRKAVTLNCLYETSWWSYYIFWYKQLPSKEMIFLIRQGSDEQNAKSGRYSVNFKKAAKSVALTISALQLEDSAKYFCALGVLPTVTGGGLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDST

SEQ ID NO: 15 - protein, 307 aa, Homo sapiens
MRWALAVLLAFLSPASQKSSNLEGRTKSVTRQTGSSAEITCDLTVTNTFYIHWYLHQEGKAPQRLLYYDVSTARDVLESGLSPGKYYTHTPRRWSWILRLQNLIENDSGVYYCATWDRRDYKKLFGSGTTLVVTDKQLDADVSPKPTIFLPSIAETKLQKAGTYLCLLEKFFPDVIKIHWQEKKSNTILGSQEGNTMKTNDTYMKFSWLTVPEKSLDKEHRCIVRHENNKNGVDQEIIFPPIKTDVITMDPKDNCSKDANDTLLLQLTNTSAYYMYLLLLLKSVVYFAIITCCLLRRTAFCCNGEKS

SEQ ID NO: 16 - protein, 290 aa, Homo sapiens
MLFSSLLCVFVAFSYSGSSVAQKVTQAQSSVSMPVRKAVTLNCLYETSWWSYYIFWYKQLPSKEMIFLIRQGSDEQNAKSGRYSVNFKKAAKSVALTISALQLEDSAKYFCALGVLPTVTGGGLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTVLGLRMLFAKTVAVNFLLTAKLFFL

SEQ ID NO: 17 - protein, 226 aa, Artificial Sequence
  The sequence of the extracellular region less the connecting peptide region of the gamma chain of a mutant TCR
SSNLEGRTKSVTRQTGSSAEITCDLTVTNTFYIHWYLHQEGKAPQRLLYYDVSTARDVLESGLSPGKYYTHTPRRWSWILRLQNLIENDSGVYYCATWDRRDYKKLFGSGTTLVVTDKQLDADVSPKPTIFLPSIAETKLQKAGTYLCLLEKFFPDVIKIHWQEKKSNTILGSQECNTMKTNDTYMKFSWLTVPEKSLDKEHRCIVRHENNKNGVDQEIIFPPIKT

SEQ ID NO: 18 - protein, 209 aa, Artificial Sequence
  The sequence of the extracellular region less the connecting peptide of the delta chain of a mutant TCR
AQKVTQAQSSVSMPVRKAVTLNCLYETSWWSYYIFWYKQLPSKEMIFLIRQGSDEQNAKSGRYSVNFKKAAKSVALTISALQLEDSAKYFCALGVLPTVTGGGLIFGKGTRVTVEPRSQPHTKPSVFVMKNGTNVACLVKEFYPKDIRINLVSSKKITEFDPCIVISPSGKYNAVKLGKYEDSNSVTCSVQHDNKTVHSTDFEVKTDST

SEQ ID NO: 19 - protein, 172 aa, Mus musculus
  misc_feature (1)..(1): Xaa can be any naturally occurring amino acid
XKRLDADISPKPTIFLPSVAETNLHKTGTYLCLLEKFFPDVIRVYWKEKDGNTILDSQEGDTLKTNDTYMKFSWLTVPERAMGKEHRCIVKHENNKGGADQEIFFPSIKKVAVSTKPTTCWQDKNDVLQLQFTITSAYYTYLLLLLKSVIYLAIISFSLLRRTSVCGNEKKS

SEQ ID NO: 20 - protein, 153 aa, Mus musculus
  misc_feature (1)..(1): Xaa can be any naturally occurring amino acid
XSQPPAKPSVFIMKNGTNVACLVKDFYPKEVTISLRSSKKIVEFDPAIVISPSGKYSAVKLGQYGDSNSVTCSVQHNSETVHSTDFEPYANSFNNEKLPEPENDTQISEPCYGPRVTVHTEKVNMMSLTVLGLRLLFAKTIAINFLLTVKLFF

SEQ ID NO: 21 - DNA, 921 bp, Homo sapiens
atgaggtgggccctggccgtgctcctggcttttctcagccctgcctcccagaagagctccaacctggagggaaggaccaagtccgtgaccagacagacaggcagcagcgccgagatcacctgtgatctgaccgtgaccaataccttttacatccattggtacctgcatcaggagggcaaggcccctcaaaggctgctctactacgatgtctccaccgccagagatgtgctggaatccggactgagccccggcaaatattacacccatacccccaggaggtggtcctggatcctgagactgcagaatctgatcgagaatgacagcggcgtgtactactgcgccacctgggacagaagggattacaagaagctgttcggcagcggcacaaccctggtcgtgaccgacaagcagctggacgccgatgtgagccctaagcccacaatctttctgccctccatcgctgagaccaaactgcagaaggccggcacatatctctgtctcctggagaagttttttcccgacgtgatcaagatccattggcaggagaagaagagcaacaccattctcggcagccaggaaggcaataccatgaaaacaaatgacacatacatgaagttttcctggctgacagtccccgagaaaagcctcgacaaggaacacaggtgcatcgtcaggcacgagaacaataagaacggcgtggaccaagagatcatttttcctcctatcaaaacagacgtcatcacaatggatcccaaggacaactgcagcaaggatgccaatgacaccctgctcctgcagctgaccaacaccagcgcctactacatgtacctgctgctgctcctgaagtccgtggtgtacttcgctatcatcacatgctgcctgctgagaagaaccgccttctgttgcaacggagagaagtcc

SEQ ID NO: 22 - DNA, 870 bp, Homo sapiens
atgctgttctccagcctgctgtgtgtgtttgtcgccttcagctatagcggctccagcgtggcccagaaggtgacccaggcccagtccagcgtgagcatgcccgtgaggaaggccgtcaccctgaactgtctgtacgagaccagctggtggagctactacatcttctggtacaagcagctgcccagcaaggagatgatctttctgatcaggcagggctccgatgaacagaatgccaagagcggcaggtactccgtgaacttcaagaaggccgccaaaagcgtcgccctgaccatctccgccctgcagttagaagactccgccaagtacttttgtgccctgggagtgctgcctaccgtgaccggaggcggcctgatctttggcaaaggaaccagggtcaccgtggagcctagatcccaaccccacaccaaacccagcgtgttcgtgatgaaaaacggcaccaacgtggcctgtctcgtcaaggaattttaccccaaagacatcaggatcaacctcgtgtcctccaagaagatcaccgagttcgaccctgccatcgtgatctcccctagcggaaagtacaacgccgtgaaactgggaaagtacgaggattccaactccgtgacctgctccgtccaacatgacaacaagaccgtgcacagcaccgacttcgaggtgaagacagatagcaccgatcacgtgaagcccaaggaaacagagaacaccaagcagcctagcaagtcctgccacaagcccaaggccatcgtccacaccgagaaagtcaacatgatgagcctgaccgtgctgggcctgaggatgctgtttgccaagaccgtcgccgtgaatttcctgctgaccgccaaactcttcttcctg

SEQ ID NO: 23 - DNA, 1859 bp, Artificial Sequence
  The codon-optimised DNA sequence encoding a TCR comprising the gamma and delta chains connected by a linker
atgaggtgggccctggccgtgctcctggcttttctcagccctgcctcccagaagagctccaacctggagggaaggaccaagtccgtgaccagacagacaggcagcagcgccgagatcacctgtgatctgaccgtgaccaataccttttacatccattggtacctgcatcaggagggcaaggcccctcaaaggctgctctactacgatgtctccaccgccagagatgtgctggaatccggactgagccccggcaaatattacacccatacccccaggaggtggtcctggatcctgagactgcagaatctgatcgagaatgacagcggcgtgtactactgcgccacctgggacagaagggattacaagaagctgttcggcagcggcacaaccctggtcgtgaccgacaagcagctggacgccgatgtgagccctaagcccacaatctttctgccctccatcgctgagaccaaactgcagaaggccggcacatatctctgtctcctggagaagttttttcccgacgtgatcaagatccattggcaggagaagaagagcaacaccattctcggcagccaggaaggcaataccatgaaaacaaatgacacatacatgaagttttcctggctgacagtccccgagaaaagcctcgacaaggaacacaggtgcatcgtcaggcacgagaacaataagaacggcgtggaccaagagatcatttttcctcctatcaaaacagacgtcatcacaatggatcccaaggacaactgcagcaaggatgccaatgacaccctgctcctgcagctgaccaacaccagcgcctactacatgtacctgctgctgctcctgaagtccgtggtgtacttcgctatcatcacatgctgcctgctgagaagaaccgccttctgttgcaacggagagaagtccggatccggatccggcgagggacgcggaagcctgctgacctgtggcgacgtggaggaaaaccccgggcccatgctgttctccagcctgctgtgtgtgtttgtcgccttcagctatagcggctccagcgtggcccagaaggtgacccaggcccagtccagcgtgagcatgcccgtgaggaaggccgtcaccctgaactgtctgtacgagaccagctggtggagctactacatcttctggtacaagcagctgcccagcaaggagatgatctttctgatcaggcagggctccgatgaacagaatgccaagagcggcaggtactccgtgaacttcaagaaggccgccaaaagcgtcgccctgaccatctccgccctgcagttagaagactccgccaagtacttttgtgccctgggagtgctgcctaccgtgaccggaggcggcctgatctttggcaaaggaaccagggtcaccgtggagcctagatcccaaccccacaccaaacccagcgtgttcgtgatgaaaaacggcaccaacgtggcctgtctcgtcaaggaattttaccccaaagacatcaggatcaacctcgtgtcctccaagaagatcaccgagttcgaccctgccatcgtgatctcccctagcggaaagtacaacgccgtgaaactgggaaagtacgaggattccaactccgtgacctgctccgtccaacatgacaacaagaccgtgcacagcaccgacttcgaggtgaagacagatagcaccgatcacgtgaagcccaaggaaacagagaacaccaagcagcctagcaagtcctgccacaagcccaaggccatcgtccacaccgagaaagtcaacatgatgagcctgaccgtgctgggcctgaggatgctgtttgccaagaccgtcgccgtgaatttcctgctgaccgccaaactcttcttcct

SEQ ID NO: 24 - protein, 619 aa, Artificial Sequence
  The polypeptide encoded by the DNA sequence of SEQ ID NO 23
MRWALAVLLAFLSPASQKSSNLEGRTKSVTRQTGSSAEITCDLTVTNTFYIHWYLHQEGKAPQRLLYYDVSTARDVLESGLSPGKYYTHTPRRWSWILRLQNLIENDSGVYYCATWDRRDYKKLFGSGTTLVVTDKQLDADVSPKPTIFLPSIAETKLQKAGTYLCLLEKFFPDVIKIHWQEKKSNTILGSQEGNTMKTNDTYMKFSWLTVPEKSLDKEHRCIVRHENNKNGVDQEIIFPPIKTDVITMDPKDNCSKDANDTLLLQLTNTSAYYMYLLLLLKSVVYFAIITCCLLRRTAFCCNGEKSGSGSGEGRGSLLTCGDVEENPGPMLFSSLLCVFVAFSYSGSSVAQKVTQAQSSVSMPVRKAVTLNCLYETSWWSYYIFWYKQLPSKEMIFLIRQGSDEQNAKSGRYSVNFKKAAKSVALTISALQLEDSAKYFCALGVLPTVTGGGLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTVLGLRMLFAKTVAVNFLLTAKLFF

SEQ ID NO: 25 - protein, 5 aa, Artificial Sequence
  The sequence of a linker
GGGGS

SEQ ID NO: 26 - protein, 10 aa, Artificial Sequence
  The sequence of a purification tag
EQKLISEEDL

SEQ ID NO: 27 - protein, 8 aa, Artificial Sequence
  The sequence of a purification tag
DYKDDDDK

SEQ ID NO: 28 - protein, 9 aa, Artificial Sequence
  The sequence of a purification tag
YPYDVPDYA

SEQ ID NO: 29 - protein, 669 aa, Homo sapiens
MEGNKLEEQDSSPPQSTPGLMKGNKREEQGLGPEPAAPQQPTAEEEALIEFHRSYRELFEFFCNNTTIHGAIRLVCSQHNRMKTAFWAVLWLCTFGMMYWQFGLLFGEYFSYPVSLNINLNSDKLVFPAVTICTLNPYRYPEIKEELEELDRITEQTLFDLYKYSSFTTLVAGSRSRRDLRGTLPHPLQRLRVPPPPHGARRARSVASSLRDNNPQVDWKDWKIGFQLCNQNKSDCFYQTYSSGVDAVREWYRFHYINILSRLPETLPSLEEDTLGNFIFACRFNQVSCNQANYSHFHHPMYGNCYTFNDKNNSNLWMSSMPGINNGLSLMLRAEQNDFIPLLSTVTGARVMVHGQDEPAFMDDGGFNLRPGVETSISMRKETLDRLGGDYGDCTKNGSDVPVENLYPSKYTQQVCIHSCFQESMIKECGCAYIFYPRPQNVEYCDYRKHSSWGYCYYKLQVDFSSDHLGCFTKCRKPCSVTSYQLSAGYSRWPSVTSQEWVFQMLSRQNNYTVNNKRNGVAKVNIFFKELNYKTNSESPSVTMVTLLSNLGSQWSLWFGSSVLSVVEMAELVFDLLVIMFLMLLRRFRSRYWSPGRGGRGAQEVASTLASSPPSHFCPHPMSLSLSQPGPAPSPALTAPPPAYATLGPRPSPGGSAGASSSTCPLGGP

SEQ ID NO: 30 - protein, 728 aa, Homo sapiens
MGMARGSLTRVPGVMGEGTQGPELSLDPDPCSPQSTPGLMKGNKLEEQDPRPLQPIPGLMEGNKLEEQDSSPPQSTPGLMKGNKREEQGLGPEPAAPQQPTAEEEALIEFHRSYRELFEFFCNNTTIHGAIRLVCSQHNRMKTAFWAVLWLCTFGMMYWQFGLLFGEYFSYPVSLNINLNSDKLVFPAVTICTLNPYRYPEIKEELEELDRITEQTLFDLYKYSSFTTLVAGSRSRRDLRGTLPHPLQRLRVPPPPHGARRARSVASSLRDNNPQVDWKDWKIGFQLCNQNKSDCFYQTYSSGVDAVREWYRFHYINILSRLPETLPSLEEDTLGNFIFACRFNQVSCNQANYSHFHHPMYGNCYTFNDKNNSNLWMSSMPGINNGLSLMLRAEQNDFIPLLSTVTGARVMVHGQDEPAFMDDGGFNLRPGVETSISMRKETLDRLGGDYGDCTKNGSDVPVENLYPSKYTQQVCIHSCFQESMIKECGCAYIFYPRPQNVEYCDYRKHSSWGYCYYKLQVDFSSDHLGCFTKCRKPCSVTSYQLSAGYSRWPSVTSQEWVFQMLSRQNNYTVNNKRNGVAKVNIFFKELNYKTNSESPSVTMVTLLSNLGSQWSLWFGSSVLSVVEMAELVFDLLVIMFLMLLRRFRSRYWSPGRGGRGAQEVASTLASSPPSHFCPHPMSLSLSQPGPAPSPALTAPPPAYATLGPRPSPGGSAGASSSTCPLGGP

SEQ ID NO: 31 - protein, 245 aa, Homo sapiens
MEGNKLEEQDSSPPQSTPGLMKGNKREEQGLGPEPAAPQQPTAEEEALIEFHRSYRELFEFFCNNTTIHGAIRLVCSQHNRMKTAFWAVLWLCTFGMMYWQFGLLFGEYFSYPVSLNINLNSDKLVFPAVTICTLNPYRYPEIKEELEELDRITEQTLFDLYKYSSFTTLVAGSRSRRDLRGTLPHPLQRLRVPPPPHGARRARSVASSLRDNNPQVDWKDWKIGFQLELLSLPPPDVWKLLYFG

SEQ ID NO: 32 - protein, 650 aa, Homo sapiens
MEGNKLEEQDSSPPQSTPGLMKGNKREEQGLGPEPAAPQQPTAEEEALIEFHRSYRELFEFFCNNTTIHGAIRLVCSQHNRMKTAFWAVLWLCTFGMMYWQFGLLFGEYFSYPVSLNINLNSDKLVFPAVTICTLNPYRYPEIKEELEELDRITEQTLFDLYKYSSFTTLVAGSRSRRDLRGTLPHPLQRLRVPPPPHGARRARSVASSLRDNNPQVDWKDWKIGFQLCNQNKSDCFYQTYSSGVDAVREWYRFHYINILSRLPETLPSLEEDTLGNFIFACRFNQVSCNQANYSHFHHPMYGNCYTFNDKNNSNLWMSSMPGINNVTGARVMVHGQDEPAFMDDGGFNLRPGVETSISMRKETLDRLGGDYGDCTKNGSDVPVENLYPSKYTQQVCIHSCFQESMIKECGCAYIFYPRPQNVEYCDYRKHSSWGYCYYKLQVDFSSDHLGCFTKCRKPCSVTSYQLSAGYSRWPSVTSQEWVFQMLSRQNNYTVNNKRNGVAKVNIFFKELNYKTNSESPSVTMVTLLSNLGSQWSLWFGSSVLSVVEMAELVFDLLVIMFLMLLRRFRSRYWSPGRGGRGAQEVASTLASSPPSHFCPHPMSLSLSQPGPAPSPALTAPPPAYATLGPRPSPGGSAGASSSTCPLGGP

SEQ ID NO: 33 - protein, 691 aa, Homo sapiens
MEGNKLEEQDSSPPQSTPGLMKGNKREEQGLGPEPAAPQQPTAEEEALIEFHRSYRELFEFFCNNTTIHGAIRLVCSQHNRMKTAFWAVLWLCTFGMMYWQFGLLFGEYFSYPVSLNINLNSDKLVFPAVTICTLNPYRYPEIKEELEELDRITEQTLFDLYKYSSFTTLVAGSRSRRDLRGTLPHPLQRLRVPPPPHGARRARSVASSLRDNNPQVDWKDWKIGFQLCNQNKSDCFYQTYSSGVDAVREWYRFHYINILSRLPETLPSLEEDTLGNFIFACRFNQVSCNQANYSHFHHPMYGNCYTFNDKNNSNLWMSSMPGINNGLSLMLRAEQNDFIPLLSTVTGARVMVHGQDEPAFMDDGGFNLRPGVETSISMRKETLDRLGGDYGDCTKNGSDVPVENLYPSKYTQQVCIHSCFQESMIKECGCAYIFYPRPQNVEYCDYRKHSSWGQVRSLTPVIPALWEAEAGGSRGYCYYKLQVDFSSDHLGCFTKCRKPCSVTSYQLSAGYSRWPSVTSQEWVFQMLSRQNNYTVNNKRNGVAKVNIFFKELNYKTNSESPSVTMVTLLSNLGSQWSLWFGSSVLSVVEMAELVFDLLVIMFLMLLRRFRSRYWSPGRGGRGAQEVASTLASSPPSHFCPHPMSLSLSQPGPAPSPALTAPPPAYATLGPRPSPGGSAGASSSTCPLGGP

SEQ ID NO: 34 - protein, 692 aa, Homo sapiens
MSSIKGNKLEEQDPRPLQPIPGLMEGNKLEEQDSSPPQSTPGLMKGNKREEQGLGPEPAAPQQPTAEEEALIEFHRSYRELFEFFCNNTTIHGAIRLVCSQHNRMKTAFWAVLWLCTFGMMYWQFGLLFGEYFSYPVSLNINLNSDKLVFPAVTICTLNPYRYPEIKEELEELDRITEQTLFDLYKYSSFTTLVAGSRSRRDLRGTLPHPLQRLRVPPPPHGARRARSVASSLRDNNPQVDWKDWKIGFQLCNQNKSDCFYQTYSSGVDAVREWYRFHYINILSRLPETLPSLEEDTLGNFIFACRFNQVSCNQANYSHFHHPMYGNCYTFNDKNNSNLWMSSMPGINNGLSLMLRAEQNDFIPLLSTVTGARVMVHGQDEPAFMDDGGFNLRPGVETSISMRKETLDRLGGDYGDCTKNGSDVPVENLYPSKYTQQVCIHSCFQESMIKECGCAYIFYPRPQNVEYCDYRKHSSWGYCYYKLQVDFSSDHLGCFTKCRKPCSVTSYQLSAGYSRWPSVTSQEWVFQMLSRQNNYTVNNKRNGVAKVNIFFKELNYKTNSESPSVTMVTLLSNLGSQWSLWFGSSVLSVVEMAELVFDLLVIMFLMLLRRFRSRYWSPGRGGRGAQEVASTLASSPPSHFCPHPMSLSLSQPGPAPSPALTAPPPAYATLGPRPSPGGSAGASSSTCPLGGP

SEQ ID NO: 35 - protein, 226 aa, Artificial Sequence
  The sequence of the extracellular region less the connecting peptide region of the gamma chain of a mutant TCR
SSNLEGRTKSVTRQTGSSAEITCDLTVTNTFYIHWYLHQEGKAPQRLLYYDVSTARDVLESGLSPGKYYTHTPRRWSWILRLQNLIENDSGVYYCATWDRRDYKKLFGSGTTLVVTDKQLDADVSPKPTIFLPSIAETKLQKAGTYLCLLEKFFPDVIKIHWQEKKSNTILGCQEGNTMKTNDTYMKFSWLTVPEKSLDKEHRCIVRHENNKNGVDQEIIFPPIKT

SEQ ID NO: 36 - protein, 5 aa, Artificial Sequence
  The sequence of a linking peptide
GSGSG

SEQ ID NO: 37 - protein, 18 aa, Artificial Sequence
  The sequence of a T2A self-cleaving linker
EGRGSLLTCGDVEENPGP

(19) United States
(12) Patent Application Publication
(10) Pub. No.: US 2021/0403528 A1
(43) Pub. Date: Dec. 30, 2021

(54) Novel T-Cell Receptor and Ligand

(71) Applicant: University College Cardiff Consultants Ltd., Cardiff South Glamorgan (GB)

(72) Inventors: Andrew SEWELL, Cardiff South Glamorgan (GB); Garry DOLTON, Cardiff South Glamorgan (GB)

(21) Appl. No.: 17/326,661

(22) Filed: May 21, 2021

Related U.S. Application Data
(60) Provisional application No. 63/028,338, filed on May 21, 2020.

(51) Int. Cl. C07K 14/725; A61K 35/17; C12N 15/86

(52) U.S. Cl. CPC C07K 14/7051; A61K 35/17; A61K 38/00; C07K 2317/565; C12N 15/86

(57) ABSTRACT

The present disclosure relates to a new T-cell receptor (TCR), in particular at least one complementarity-determining region (CDR) thereof; a T-cell expressing said TCR; a clone expressing said TCR; a vector encoding said TCR; a soluble version of said TCR; a pharmaceutical composition or bispecific comprising said TCR, said cell, said clone or said vector; use of said TCR or said cell or said clone or said vector or said pharmaceutical composition or bispecific to treat cancer; and a method of treating cancer using said TCR, said cell, said clone, said vector, said pharmaceutical composition or bispecific comprising said TCR.

### CROSS-REFERENCE TO RELATED APPLICATIONS

This application claims the benefit of U.S. Provisional Application No. 63/028,338, filed May 21, 2020, the disclosure of which is hereby incorporated by reference in its entirety.

### TECHNICAL FIELD

The present disclosure relates to a new T-cell receptor (TCR), in particular at least one complementarity-determining region (CDR) thereof; a T-cell expressing said TCR; a clone expressing said TCR; a vector encoding said TCR; a soluble version of said TCR; a pharmaceutical composition or bispecific comprising said TCR, said cell, said clone or said vector; use of said TCR or said cell or said clone or said vector or said pharmaceutical composition or bispecific to treat cancer; a method of treating cancer using said TCR, said cell, said clone, said vector, said pharmaceutical composition or bispecific comprising said TCR; and a ligand with which said TCR binds.

### BACKGROUND

We have discovered a new type of γδ T-cell effective for treating cancer, which requires that the target cell expresses an intact SCNN1A gene product for recognition. The SCNN1A gene encodes for the alpha subunit of a protein complex called the epithelial sodium channel (ENaC). This new type of T-cell does not follow the convention of requiring a specific Human Leukocyte Antigen (HLA) for target recognition and is therefore said to be ‘unconventional’. The HLA locus is highly variable with over 17,000 different alleles having been described today. As such, any therapeutic approach that works via an HLA may only be effective in a minority of patients. In contrast, it is thought that the entire human population expresses SCNN1A, the gene required for recognition of cancer cells via our γδ T cell receptor (TCR) and its corresponding new T-cell clone, hereinafter termed SW.3G1. This clone was discovered during a screen for γδ T-cells that could recognize Lymphoblastoid Cell Lines (LCLs) created by infecting healthy B-cells with Epstein-Barr virus (EBV) also called human herpesvirus 4 (HHV-4). Advantageously, the SW.3G1 γδ T-cell clone does not respond to healthy B-cells or other healthy cell lines.

Further studies have shown that the SW.3G1 γδ T-cell clone can recognize most, if not all, cancer cells. The SCNN1A gene product is required for this recognition and thus is the binding ligand for the SW.3G1 TCR.

A γδ TCR is a disulfide-linked membrane-anchored heterodimeric protein normally consisting of the highly variable gamma and delta chains that associate with the invariant CD3 chain molecules to form a complete functioning TCR. T cells expressing this receptor are referred to as γ:δ (or γδ) T cells.

The γ and δ chains are composed of extracellular domains comprising a Constant (C) region and a Variable (V) region. The Constant region is proximal to the cell membrane, followed by a transmembrane region and a short cytoplasmic tail, while the Variable region binds to the ligand. The ligands for most γδ T cells remain unknown.

The variable domain is formed of variable regions of both the TCR γ chain and δ chain each of which has three hypervariable regions called complementarity determining regions (CDRs). In general, the antigen-binding site is formed by the CDR loops of the TCR γ chain and δ chain. CDR1γ and CDR2γ are encoded by the individual Vγ genes whereas CDR1δ and CDR2δ are encoded by the individual VO genes. The CDR3 of the TCR γ chain is hypervariable due to the potential for nucleotide addition and removal around the joining of the V region and a Joining region. The TCR δ chain CDR3 has even more capacity for variation and is especially hypervariable as it can also include a diversity (D) gene after VDJ recombination has occurred.

In 2015 about 90.5 million people had cancer. About 14.1 million new cases occur a year (not including skin cancer other than melanoma). It causes about 8.8 million deaths (15.7%) of human deaths. The most common types of cancer in males are lung cancer, prostate cancer, colorectal cancer and stomach cancer. In females, the most common types of cancer are breast cancer, colorectal cancer, lung cancer and cervical cancer. If skin cancer, other than melanoma, were included in total new cancers each year it would account for around 40% of cases. In children, acute lymphoblastic leukaemia and brain tumours are most common except in Africa where non-Hodgkin lymphoma occurs more often. In 2012, about 165,000 children under 15 years of age were diagnosed with cancer.

The risk of cancer increases significantly with age and many cancers occur more commonly in developed countries. Rates are increasing as more people live to an old age and as lifestyle changes occur in the developing world. The financial costs of cancer were estimated at $1.16 trillion per year as of 2010. It follows that there is a need to provide better and safer ways of treating or eradicating this disease. An immunotherapy that uses the body's natural defence systems to kill aberrant tissue is acknowledged to be safer than chemical intervention but, to be effective, the immunotherapy must be cancer specific. Moreover, the discovery of an immunotherapy that is effective against many types of cancer would be extremely beneficial as not only could it be administered to individuals suffering from many different types of cancer (i.e. it would have pan-population application) but it could also be administered to a single individual suffering from more than one type of cancer. Additionally, the identification of an immunotherapy that was not HLA-restricted would also be extremely advantageous as it means it could be administered to any individual regardless of HLA tissue type.

### BRIEF SUMMARY OF THE INVENTION

The T cells we have identified herein have the aforementioned advantageous characteristics in that they are effective against many types of cancer and they are not HLA-restricted. They therefore have pan-population application due to the ubiquitous expression of the SCNN1A gene product that is required for recognition.

According to a first aspect of the invention there is provided a tumour specific T-cell receptor (TCR) comprising a CDR comprising or consisting of CATWDRRDYKKLF (CDR3γ, SEQ ID NO: 1) and/or a CDR comprising or consisting of CALGVLPTVTGGGLIF (CDR3δ, SEQ ID NO: 2).

### BRIEF DESCRIPTION OF THE SEQUENCES

SEQ ID NO: 1 is CDR3 of the γ chain of a TCR according to the invention.

SEQ ID NO: 2 is CDR3 of the δ chain of a TCR according to the invention.

SEQ ID NO: 3 is CDR1 of the γ chain of a TCR according to the invention.

SEQ ID NO: 4 is CDR2 of the γ chain of a TCR according to the invention.

SEQ ID NO: 5 is CDR1 of the δ chain of a TCR according to the invention.

SEQ ID NO: 6 is CDR2 of the δ chain of a TCR according to the invention.

SEQ ID NO: 7 is the sequence of the extracellular region less the connecting peptide region of the native γ chain of the TCR of clone SW.3G1 according to the invention.

SEQ ID NO: 8 is the sequence of the extracellular region less the connecting peptide region of the δ chain of a mutant TCR according to the invention.

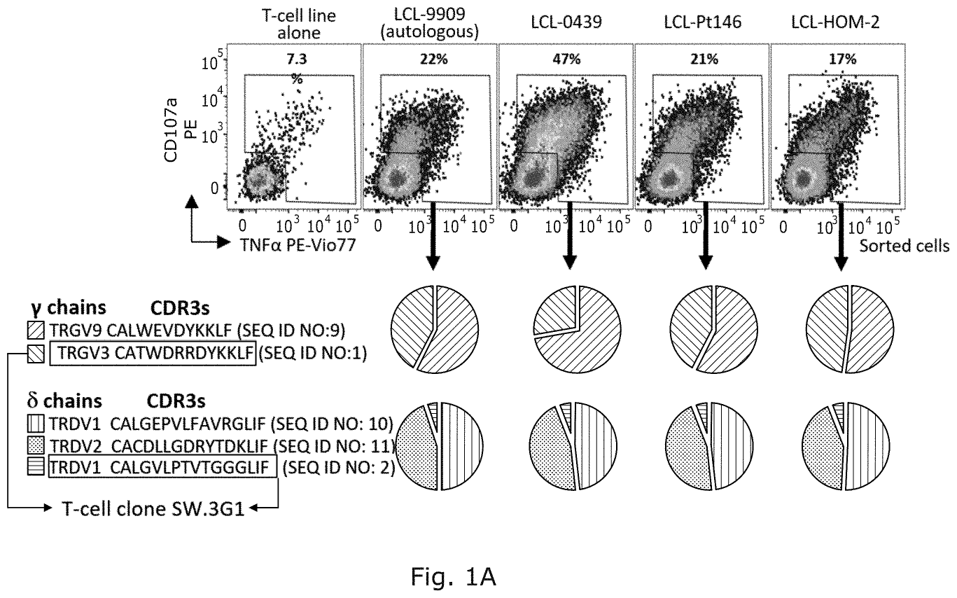

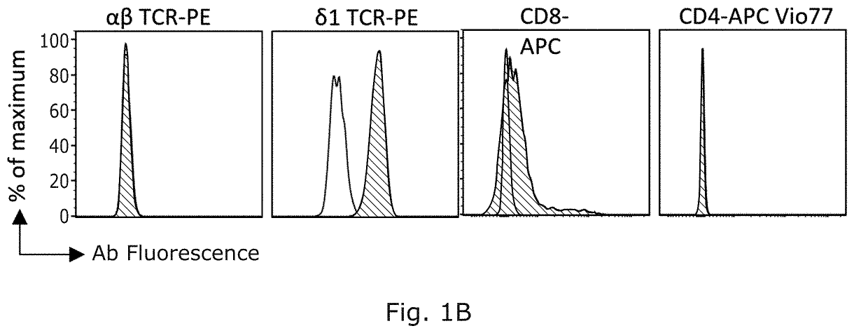

SEQ ID NO: 9 is CDR3 of the γ chain from TRGV9 of a TCR shown in FIG. 1A

SEQ ID NO: 10 is CDR3 of the δ chain from TRDV1 of a TCR shown in FIG. 1A

SEQ ID NO: 11 is CDR3 of the δ chain from TRDV2 of a TCR shown in FIG. 1A

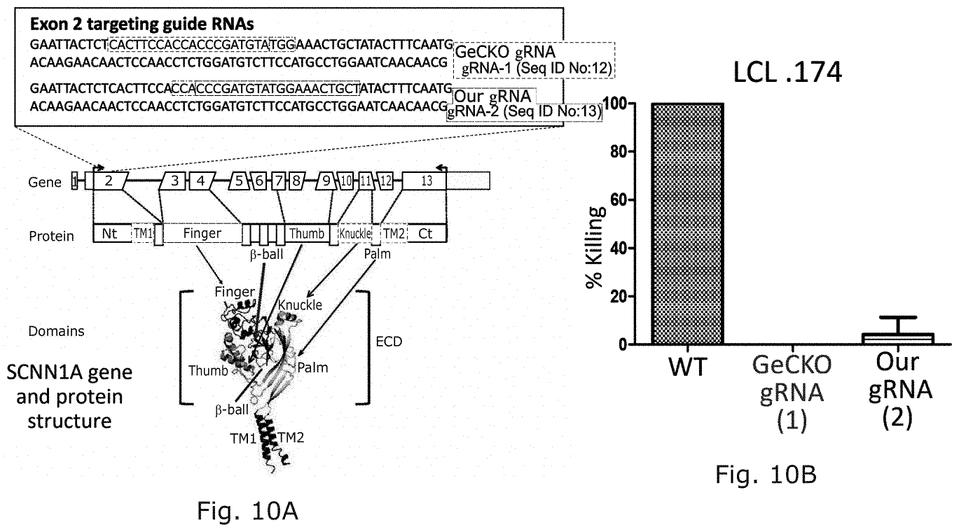

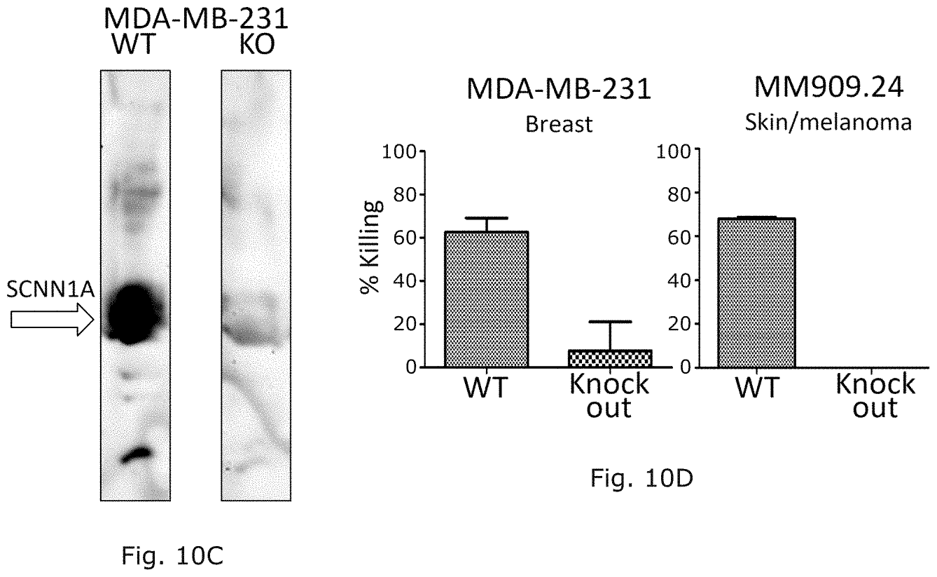

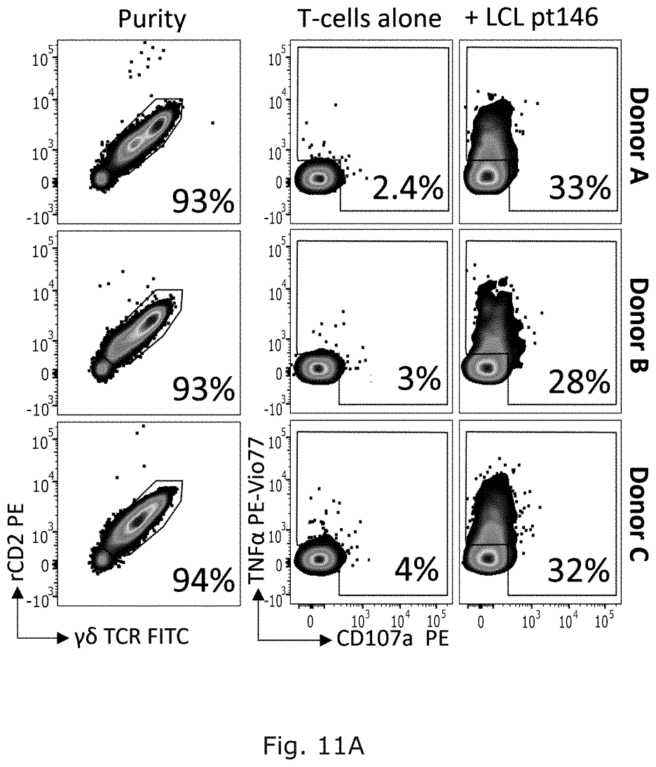

SEQ ID NO: 12 is a GeCKO gRNA sequence shown in FIG. 10A

SEQ ID NO: 13 is a gRNA sequence shown in FIG. 10A

SEQ ID NO: 14 is the sequence of the extracellular region less the connecting peptide region of the native δ chain of the TCR of clone SW.3G1 according to the invention

SEQ ID NO: 15 is the sequence of the native γ chain of the TCR of clone SW.3G1 according to the invention

SEQ ID NO: 16 is the sequence of the native δ chain of the TCR of clone SW.3G1 according to the invention

SEQ ID NO: 17 is the sequence of the extracellular region less the connecting peptide region of the γ chain of a mutant TCR according to the invention.

SEQ ID NO: 18 is the sequence of the extracellular region less the connecting peptide of the δ chain of a mutant TCR according to the invention.

SEQ ID NO: 19 is the sequence of a murine constant region of the γ chain of a TCR.

SEQ ID NO: 20 is the sequence of a murine constant region of the δ chain of a TCR.

SEQ ID NO: 21 is the native DNA sequence encoding the native γ chain of the TCR of clone SW.3G1 according to the invention

SEQ ID NO: 22 is the native DNA sequence encoding the native δ chain of the TCR of clone SW.3G1 according to the invention

SEQ ID NO: 23 is a codon-optimised DNA sequence encoding a TCR comprising the γ and δ chains connected by a linker (see Example 1(i))

SEQ ID NO: 24 is the polypeptide encoded by the DNA sequence of SEQ ID NO: 23 (see Example 1(i))

SEQ ID NO: 25 is the sequence of a linker (see Example 4(c)) SEQ ID NO: 26 is the sequence of a purification tag.

SEQ ID NO: 27 is the sequence of a purification tag.

SEQ ID NO: 28 is the sequence of a purification tag.

SEQ ID NO: 29 is the sequence of Isoform 1 of SCNN1A gene product

SEQ ID NO: 30 is the sequence of Isoform 2 of SCNN1A gene product

SEQ ID NO: 31 is the sequence of Isoform 3 of SCNN1A gene product

SEQ ID NO: 32 is the sequence of Isoform 4 of SCNN1A gene product

SEQ ID NO: 33 is the sequence of Isoform 5 of SCNN1A gene product

SEQ ID NO: 34 is the sequence of Isoform 6 of SCNN1A gene product

SEQ ID NO: 35 is the sequence of the extracellular region less the connecting peptide region of the γ chain of a mutant TCR according to the invention.

SEQ ID NO: 36 is the sequence of a linking peptide

SEQ ID NO: 37 is the sequence of a T2A self-cleaving linker

### DETAILED DESCRIPTION OF THE INVENTION

### Definitions

Suitably, the polypeptides and polynucleotides used in the present invention are isolated. An “isolated” polypeptide or polynucleotide is one that is removed from its original environment. For example, a naturally-occurring polypeptide or polynucleotide is isolated if it is separated from some or all of the coexisting materials in the natural system. A polynucleotide is considered to be isolated if, for example, it is cloned into a vector that is not a part of its natural environment.

“Naturally occurring” or “native”, which terms are interchangeable, when used with reference to a polypeptide or polynucleotide sequence means a sequence found in nature and not synthetically modified.

The term “artificial” when used with reference to a polypeptide or polynucleotide sequence means a sequence not found in nature which is, for example, a synthetic modification of a natural sequence, or contains an unnatural polypeptide or polynucleotide sequence.

The term “engineered” when used with reference to a cell means a cell not found in nature which is, for example, a synthetic modification of a natural cell, for example, because it contains or expresses foreign elements and/or lacks natural elements.

The term “heterologous” when used with reference to the relationship of one polynucleotide or polypeptide to another polynucleotide or polypeptide indicates that the two or more sequences are not found in the same relationship to each other in nature.

The term “heterologous” when used with reference to the relationship of one polynucleotide or polypeptide sequence to a cell means a sequence which is not isolated from, derived from, expressed by, associated with or based upon a naturally occurring polynucleotide or polypeptide sequence found in the said cell.

The term “domain” is generally used to refer to a part of the TCR formed of the corresponding region of the two chains. For example, the transmembrane regions of the γ and δ chains form the transmembrane domain.

The term “intracellular” domain or region is used interchangeably with the term “cytoplasmic” domain or region and in the literature this is sometimes referred to as the “cytosolic” domain or region.

T-Cell Receptor (TCR)

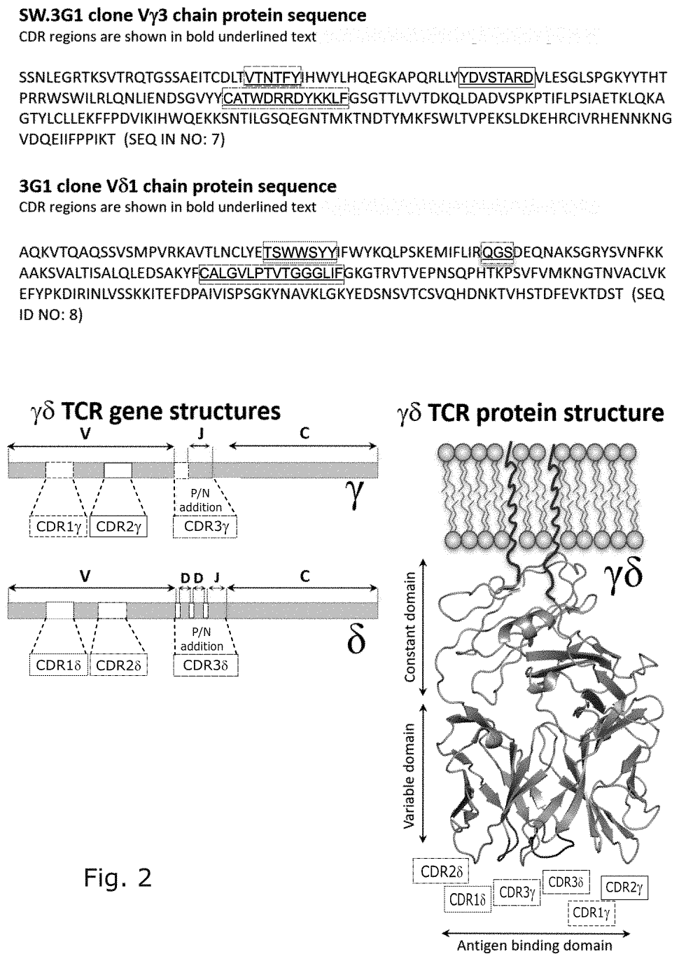

A TCR of this invention comprises a γ chain and a δ chain. The extracellular region of each chain comprises three CDRs (CDR1, CDR2, CDR3) and four framework regions which are either side of the CDRs, and a constant region. The soluble form of the TCR consists of the extracellular domain (also called the antigen-binding domain—see FIG. 2). An entire TCR (as opposed to the soluble form) has the extracellular domain as well as, at its C terminus, transmembrane and intracellular domains. An immature form of each TCR chain also comprises a leader peptide sequence at its N terminus which is removed after translation by cellular peptidases such as signal peptidase. Each chain has a connecting peptide region which links the transmembrane and intracellular regions to the extracellular domain at its C terminus.

In a preferred embodiment of the invention, said γ chain CDR3 comprises or consists of CATWDRRDYKKLF (SEQ ID NO: 1) or a CDR that shares at least 88% sequence identity therewith, such as 89%, 90%, 91%, 92%, 93%, 94%, 95%, 96%, 97%, 98% or 99% sequence identity. In a preferred embodiment of the invention, said δ chain CDR3 comprises or consists of CALGVLPTVTGGGLIF (SEQ ID NO: 2) or a CDR that shares at least 88% identity therewith, such as 89%, 90%, 91%, 92%, 93%, 94%, 95%, 96%, 97%, 98% or 99% sequence identity.

Tumour-specific binding fragments of the said TCR and the respective γ and δ chains are also provided.

As used herein, the term “tumour” will be understood to embrace solid tumours and liquid tumours (including blood cancers).

Thus, an embodiment of the invention provides a tumour-specific T-cell receptor (TCR) or a γ chain thereof or a tumour-specific binding fragment of a TCR which binds a tumour antigen comprising:
a CDR3 comprising or consisting of CATWDRRDYKKLF (SEQ ID NO: 1) or a variant CDR3 which has at least 88% sequence identity to the CDR3 of SEQ ID NO: 1 (e.g. at least 89%, 90%, 91%, 92%, 93%, 94%, 95%, 96%, 97%, 98% or 99% sequence identity therewith).

An embodiment of the invention also provides a tumour-specific T-cell receptor (TCR) or a δ chain thereof or a tumour-specific binding fragment of a TCR which binds a tumour antigen comprising:
a CDR3 comprising or consisting of CALGVLPTVTGGGLIF (SEQ ID NO: 2) or a variant CDR3 which has at least 88% sequence identity to the CDR3 of SEQ ID NO: 2 (e.g. at least 89%, 90%, 91%, 92%, 93%, 94%, 95%, 96%, 97%, 98% or 99% sequence identity therewith).

A further embodiment of the invention provides tumour specific T-cell receptor (TCR) or tumour specific binding fragment of a TCR as above wherein the TCR or tumour specific binding fragment comprises:

a γ chain CDR3 comprising or consisting of CATWDRRDYKKLF (SEQ ID NO: 1) or a variant CDR3 which has at least 88% sequence identity to the CDR3 of SEQ ID NO: 1 (e.g. at least 89%, 90%, 91%, 92%, 93%, 94%, 95%, 96%, 97%, 98% or 99% sequence identity therewith); and
a δ chain CDR3 comprising or consisting of CALGVLPTVTGGGLIF (SEQ ID NO: 2) or a variant CDR3 which has at least 88% sequence identity to the CDR3 of SEQ ID NO: 2 (e.g. at least 89%, 90%, 91%, 92%, 93%, 94%, 95%, 96%, 97%, 98% or 99% sequence identity therewith).

A further embodiment of the invention provides a tumour-specific T-cell receptor (TCR) or a tumour-specific binding fragment of a TCR as above comprising:
a γ chain CDR3 comprising or consisting of CATWDRRDYKKLF (SEQ ID NO: 1); and/or
a δ chain CDR3 comprising or consisting of CALGVLPTVTGGGLIF (SEQ ID NO: 2).

A further embodiment of the invention provides a tumour-specific T-cell receptor (TCR) or a tumour-specific binding fragment of a TCR as above comprising:
a γ chain CDR3 comprising or consisting of CATWDRRDYKKLF (SEQ ID NO: 1); and
a δ chain CDR3 comprising or consisting of CALGVLPTVTGGGLIF (SEQ ID NO: 2).

The CDRs described herein represent the CDR3s of said TCR which are the main CDRs responsible for recognizing processed antigen or ligand. The other CDRs (CDR1γ, CDR2γ, CDR1δ and CDR2δ) are encoded by the germline. Therefore, the invention further concerns a TCR also including one or more of these other CDRs i.e. CDR1γ, CDR2γ, CDR1δ and/or CDR2δ in combination with the said one or more CDR3 sequences.

Accordingly, in a preferred embodiment said TCR or tumour-specific binding fragment of a TCR wherein the TCR or tumour specific binding fragment comprises one or more, including any combination of, CDRs comprising or consisting of the following CDRs:

(CDR1γ)
SEQ ID NO: 3
VTNTFY;
(CDR2γ)
SEQ ID NO: 4
YDVSTARD;
(CDR1δ)
SEQ ID NO: 5
TSWWSYY;
and
(CDR2δ)
SEQ ID NO: 6
QGS.

Reference herein to a tumour-specific TCR is to a TCR that specifically recognises a tumour cell or a tumour cell ligand, in the context of SCNN1A gene expression, and is activated by same but is not activated by a non-tumour cell.

In a preferred embodiment of the invention said TCR is a γδ TCR having a γ chain and a δ chain and said CDR of said γ chain comprises or consists of the CDR3: CATWDRRDYKKLF (SEQ ID NO: 1) or a CDR3 that shares at least 88% identity therewith, such as 89%, 90%, 91%, 92%, 93%, 94%, 95%, 96%, 97%, 98% or 99%; and said CDR3 of said δ chain comprises or consists of the CDR3: CALGVLPTVTGGGLIF (SEQ ID NO: 2) or a CDR3 that shares at least 88% identity therewith, such as 89%, 90%, 91%, 92%, 93%, 94%, 95%, 96%, 97%, 98% or 99%. Accordingly, said TCR may comprise one or both of the afore CDRs and in a preferred embodiment comprises both of said CDRs.

In an embodiment, the tumour-specific T-cell receptor (TCR) or a tumour-specific binding fragment of a TCR comprises a γ chain with CDRs comprising or consisting of:
CATWDRRDYKKLF (SEQ ID NO: 1) or a variant CDR3 which has at least 88% sequence identity to the CDR3 of SEQ ID NO: 1 (e.g. at least 89%, 90%, 91%, 92%, 93%, 94%, 95%, 96%, 97%, 98% or 99% sequence identity therewith);

(SEQ ID NO: 3)
VTNTFY;
and
(SEQ ID NO: 4)
YDVSTARD;

In an embodiment, the tumour-specific T-cell receptor (TCR) or a tumour-specific binding fragment of a TCR comprises a δ chain with CDRs comprising or consisting of:
CALGVLPTVTGGGLIF (SEQ ID NO: 2) or a variant CDR3 which has at least 88% sequence identity to the CDR3 of SEQ ID NO: 2 (e.g. at least 89%, 90%, 91%, 92%, 93%, 94%, 95%, 96%, 97%, 98% or 99% sequence identity therewith);

(SEQ ID NO: 5)
TSWWSYY;
and
(SEQ ID NO: 6)
QGS.

In an embodiment, the tumour-specific T-cell receptor (TCR) or tumour-specific binding fragment of a TCR comprises 3 γ chain CDRs comprising or consisting of:
CATWDRRDYKKLF (SEQ ID NO: 1) or a variant CDR3 which has at least 88% sequence identity to the CDR3 of SEQ ID NO: 1 (e.g. at least 89%, 90%, 91%, 92%, 93%, 94%, 95%, 96%, 97%, 98% or 99% sequence identity therewith);

(SEQ ID NO: 3)
VTNTFY;
and
(SEQ ID NO: 4)
YDVSTARD;

and comprises 3 δ chain CDRs comprising or consisting of:
CALGVLPTVTGGGLIF (SEQ ID NO: 2) or a variant CDR3 which has at least 88% sequence identity to the CDR3 of SEQ ID NO: 2 (e.g. at least 89%, 90%, 91%, 92%, 93%, 94%, 95%, 96%, 97%, 98% or 99% sequence identity therewith);

(SEQ ID NO: 5)
TSWWSYY;
and
(SEQ ID NO: 6)
QGS.

As noted above, said TCR is unconventional in that it is not HLA-restricted, rather it binds to a tumour-specific ligand in the context of SCNN1A gene expression. The fact that these T-cells and their TCRs are not HLA-restricted means they have pan-population therapy potential and so represent an extremely important new cancer therapy.

The specific mechanism that allows killing of many types of cancer cells is still to be elucidated. Nevertheless, it may be hypothesized (and without being limited by theory) that the T-cell of the invention is able to bind to a surface-displayed SCNN1A gene product which is aberrant in the context of a tumour cell.

The sequence of the γ chain of the native SW.3G1 TCR clone is as follows:

(SEQ ID NO: 15)
MRWALAVLLAFLSPASQKSSNLEGRTKSVTRQTGSSAEITCDLTVTNTFY
IHWYLHQEGKAPQRLLYYDVSTARDVLESGLSPGKYYTHTPRRWSWILRL
QNLIENDSGVYYCATWDRRDYKKLFGSGTTLVVTDKQLDADVSPKPTIFL
PSIAETKLQKAGTYLCLLEKFFPDVIKIHWQEKKSNTILGSQEGNTMKTN
DTYMKFSWLTVPEKSLDKEHRCIVRHENNKNGVDQEIIFPPIKTDVITMD
PKDNCSKDANDTLLLQLTNTSAYYMYLLLLLKSVVYFAIITCCLLRRTAF
CCNGEKS

The variable region of the γ chain of the native SW.3G1 TCR clone is encoded by recombination of genes TRGV3 and TRGJ1.

In SEQ ID NO: 15, residues 1-18 are the N-terminal leader sequence (shown in underlined normal type), residues 19-44 are framework region 1, residues 45-50 are CDR1 (shown in underlined bold type), residues 51-67 are framework region 2, residues 68-75 are CDR2 (shown in underlined bold type), residues 76-112 are framework region 3, residues 113-125 are CDR3 (shown in underlined bold type), residues 126-134 are framework region 4 and residues 135-307 are the constant region (shown in italics type). Within the constant region, residues 245-276 are the connecting peptide region, residues 277-300 are the transmembrane region and residues 301-307 are the intracellular region.

The leader sequence is removed after translation of the protein in the host cell and is not part of the receptor when incorporated into a cell membrane.

The extracellular region of this native γ chain, less the connecting peptide region, is as follows:

(SEQ ID NO: 7)
SSNLEGRTKSVTRQTGSSAEITCDLTVTNTFYIHWYLHQEGKAPQRLLYY
DVSTARDVLESGLSPGKYYTHTPRRWSWILRLQNLIENDSGVYYCATWDR
RDYKKLFGSGTTLVVTDKQLDADVSPKPTIFLPSIAETKLQKAGTYLCLL
EKFFPDVIKIHWQEKKSNTILGSQEGNTMKTNDTYMKFSWLTVPEKSLDK
EHRCIVRHENNKNGVDQEIIFPPIKT

In SEQ ID NO: 7, residues 1-26 are framework region 1, residues 27-32 are CDR1 (shown in underlined bold type), residues 33-49 are framework region 2, residues 50-57 are CDR2 (shown in underlined bold type), residues 58-94 are framework region 3, residues 95-107 are CDR3 (shown in underlined bold type), residues 108-116 are framework region 4 and residues 117-226 are the constant region (shown in italics type).

The sequence of the δ chain of the native SW.3G1 TCR clone is as follows:

(SEQ ID NO: 16)
MLFSSLLCVFVAFSYSGSSVAQKVTQAQSSVSMPVRKAVTLNCLYETSWWS
YYIFWYKQLPSKEMIFLIRQGSDEQNAKSGRYSVNFKKAAKSVALTISALQ
LEDSAKYFCALGVLPTVTGGGLIFGKGTRVTVEP [symbol] SQPHTKPSVFVMKNGT
NVACLVKEFYPKDIRINLVSSKKITEFDPAIVISPSGKYNAVKLGKYEDSN
SVTCSVQHDNKTVHSTDFEVKTDSTDHVKPKETENTKQPSKSCHKPKAIVH
TEKVNMMSLTVLGLRMLFAKTVAVNFLLTAKLFFL

The variable region of the δ chain of the native SW.3G1 TCR clone is encoded by recombination of genes TRDV1 and TRDD2/3 TRDJ1.

In SEQ ID NO: 16, residues 1-20 are the N-terminal leader sequence (shown in underlined normal type), residues 21-46 are framework region 1, residues 47-53 are CDR1 (shown in underlined bold type), residues 54-70 are framework region 2, residues 71-73 are CDR2 (shown in underlined bold type), residues 74-110 are framework region 3, residues 111-126 are CDR3 (shown in underlined bold type), residues 127-136 are framework region 4 and residues 137-290 are the constant region (shown in italics type). Within the constant region, residues 230-265 are the connecting peptide region, residues 266-290 are the transmembrane region.

The leader sequence is removed after translation of the protein in the host cell and is not part of the receptor when incorporated into a cell membrane.

The extracellular region of this native δ chain less the connecting peptide region is as follows:

(SEQ ID NO: 14)
AQKVTQAQSSVSMPVRKAVTLNCLYETSWWSYYIFWYKQLPSKEMIFLIRQ
GSDEQNAKSGRYSVNFKKAAKSVALTISALQLEDSAKYFCALGVLPTVTGG
GLIFGKGTRVTVEPRSQPHTKPSVFVMKNGTNVACLVKEFYPKDIRINLVS
SKKITEFDPAIVISPSGKYNAVKLGKYEDSNSVTCSVQHDNKTVHSTDFEV
KTDST

In SEQ ID NO: 14, residues 1-26 are framework region 1, residues 27-33 are CDR1 (shown in underlined bold type), residues 34-50 are framework region 2, residues 51-53 are CDR2 (shown in underlined bold type), residues 54-90 are framework region 3, residues 91-106 are CDR3 (shown in underlined bold type), residues 107-117 are framework region 4 and residues 118-209 are the constant region (show in italics type).

In a further preferred embodiment of the invention said TCR γ chain is a mutant TCR γ chain and comprises or consists of:

(SEQ ID NO: 17)
SSNLEGRTKSVTRQTGSSAEITCDLTVTNTFYIHWYLHQEGKAPQRLLYYD
VSTARDVLESGLSPGKYYTHTPRRWSWILRLQNLIENDSGVYYCATWDRRD
YKKLFGSGTTLVVTDKQLDADVSPKPTIFLPSIAETKLQKAGTYLCLLEKF
FPDVIKIHWQEKKSNTILGSQE [symbol] NTMKTNDTYMKFSWLTVPEKSLDKEHRC
IVRHENNKNGVDQEIIFPPIKT

In SEQ ID NO: 17, residues 1-26 are framework region 1, residues 27-32 are CDR1 (shown in underlined bold type), residues 33-49 are framework region 2, residues 50-57 are CDR2 (shown in underlined bold type), residues 58-94 are framework region 3, residues 95-107 are CDR3 (shown in underlined bold type), residues 108-116 are framework region 4 and residues 117-226 are the constant region (shown in italics type). SEQ ID NO: 17 defines the extracellular region less the connecting peptide region of the mutant TCR γ chain.

SEQ ID NO: 17 is the same as SEQ ID NO: 7 except for residue 176 (bold and underlined) which is G in SEQ ID NO: 7 and C in SEQ ID NO: 17.

In a further preferred embodiment of the invention said TCR γ chain is a mutant TCR γ chain and comprises or consists of:

(SEQ ID NO: 35)
SSNLEGRTKSVTRQTGSSAEITCDLTVTNTFYIHWYLHQEGKAPQRLLYYD
VSTARDVLESGLSPGKYYTHTPRRWSWILRLQNLIENDSGVYYCATWDRRD
YKKLFGSGTTLVVTDKQLDADVSPKPTIFLPSIAETKLQKAGTYLCLLEKF
FPDVIKIHWQEKKSNTILG [symbol] QEGNTMKTNDTYMKFSWLTVPEKSLDKEHRC
IVRHENNKNGVDQEIIFPPIKT

In SEQ ID NO: 35, residues 1-26 are framework region 1, residues 27-32 are CDR1 (shown in underlined bold type), residues 33-49 are framework region 2, residues 50-57 are CDR2 (shown in underlined bold type), residues 58-94 are framework region 3, residues 95-107 are CDR3 (shown in underlined bold type), residues 108-116 are framework region 4 and residues 117-226 are the constant region (shown in italics type). SEQ ID NO: 35 defines the extracellular region less the connecting peptide region of the mutant TCR γ chain.

SEQ ID NO: 35 is the same as SEQ ID NO: 7 except for residue 173 (bold and underlined) which is G in SEQ ID NO: 7 and C in SEQ ID NO: 35.

In a further preferred embodiment of the invention said TCR δ chain is a mutant TCR δ chain and comprises or consists of:

(SEQ ID NO: 8)
AQKVTQAQSSVSMPVRKAVTLNCLYETSWWSYYIFWYKQLPSKEMIFLIRQ
GSDEQNAKSGRYSVNFKKAAKSVALTISALQLEDSAKYFCALGVLPTVTGG
GLIFGKGTRVTVEPNSQPHTKPSVFVMKNGTNVACLVKEFYPKDIRINLVS
SKKITEFDPAIVISPSGKYNAVKLGKYEDSNSVTCSVQHDNKTVHSTDFEV
KTDST

or a sequence that has at least 88% identity therewith, such as 89%, 90%, 91%, 92%, 93%, 94%, 95%, 96%, 97%, 98% or 99%.

In SEQ ID NO: 8, residues 1-26 are framework region 1, residues 27-33 are CDR1 (shown in underlined bold type), residues 34-50 are framework region 2, residues 51-53 are CDR2 (shown in underlined bold type), residues 54-90 are framework region 3, residues 91-106 are CDR3 (shown in underlined bold type), residues 107-117 are framework region 4 and residues 118-209 are the constant region (shown in italics type). SEQ ID NO: 8 defines the extracellular region less the connecting peptide region of the mutant TCR δ chain.

SEQ ID NO: 8 is the same as SEQ ID NO: 14 except for residue 117 (underlined) which is N in SEQ ID NO: 8 and R in SEQ ID NO: 14.

In a further preferred embodiment of the invention said TCR δ chain is a mutant TCR δ chain and comprises or consists of:

(SEQ ID NO: 18)
AQKVTQAQSSVSMPVRKAVTLNCLYETSWWSYYIFWYKQLPSKEMIFLIRQ
GSDEQNAKSGRYSVNFKKAAKSVALTISALQLEDSAKYFCALGVLPTVTGG
GLIFGKGTRVTVEPRSQPHTKPSVFVMKNGTNVACLVKEFYPKDIRINLVS
SKKITEFDP [symbol] IVISPSGKYNAVKLGKYEDSNSVTCSVQHDNKTVHSTDFEV
KTDST

In SEQ ID NO: 18, residues 1-26 are framework region 1, residues 27-33 are CDR1 (shown in underlined bold type), residues 34-50 are framework region 2, residues 51-53 are CDR2 (shown in underlined bold type), residues 54-90 are framework region 3, residues 91-106 are CDR3 (shown in underlined bold type), residues 107-117 are framework region 4 and residues 118-209 are the constant region (show in italics type). SEQ ID NO: 18 defines the extracellular region less the connecting peptide region of the mutant TCR δ chain.

SEQ ID NO: 18 is the same as SEQ ID NO: 14 except for residue 163 (underlined) which is A in SEQ ID NO: 14 and C in SEQ ID NO: 18.

In an embodiment, the tumour-specific T-cell receptor (TCR) or tumour-specific binding fragment of a TCR comprises a γ chain extracellular region less the connecting peptide region comprising or consisting of SEQ ID NO: 7; or a variant γ chain extracellular region less the connecting peptide which has at least 88% sequence identity to the γ chain extracellular region less the connecting peptide region of SEQ ID NO: 7 (e.g. at least 89%, 90%, 91%, 92%, 93%, 94%, 95%, 96%, 97%, 98% or 99% sequence identity therewith).

An example of a variant of SEQ ID NO: 7 is SEQ ID NO: 17 or SEQ ID NO: 35.

In an embodiment, the tumour-specific T-cell receptor (TCR) or tumour-specific binding fragment of a TCR comprises a γ chain extracellular region less the connecting peptide region comprising or consisting of SEQ ID NO: 17; or a variant γ chain extracellular region less the connecting peptide which has at least 88% sequence identity to the γ chain extracellular region less the connecting peptide region of SEQ ID NO: 17 (e.g. at least 89%, 90%, 91%, 92%, 93%, 94%, 95%, 96%, 97%, 98% or 99% sequence identity therewith).

An example of a variant of SEQ ID NO: 17 is SEQ ID NO: 7 or SEQ ID NO: 35.

In an embodiment, the tumour-specific T-cell receptor (TCR) or tumour-specific binding fragment of a TCR comprises a γ chain extracellular region less the connecting peptide region comprising or consisting of SEQ ID NO: 35; or a variant γ chain extracellular region less the connecting peptide which has at least 88% sequence identity to the γ chain extracellular region less the connecting peptide region of SEQ ID NO: 35 (e.g. at least 89%, 90%, 91%, 92%, 93%, 94%, 95%, 96%, 97%, 98% or 99% sequence identity therewith).

An example of a variant of SEQ ID NO: 35 is SEQ ID NO: 7 or SEQ ID NO: 17.

In an embodiment, the tumour-specific T-cell receptor (TCR) or tumour-specific binding fragment of a TCR comprises a γ chain extracellular region less the connecting peptide region comprising or consisting of SEQ ID NO:7.

In an embodiment, the tumour-specific T-cell receptor (TCR) or tumour-specific binding fragment of a TCR comprises a γ chain extracellular region less the connecting peptide region comprising or consisting of SEQ ID NO: 17.

In an embodiment, the tumour-specific T-cell receptor (TCR) or tumour-specific binding fragment of a TCR comprises a γ chain extracellular region less the connecting peptide region comprising or consisting of SEQ ID NO: 35.

In an embodiment, the tumour-specific T-cell receptor (TCR) or tumour-specific binding fragment of a TCR comprises a δ chain extracellular region less the connecting peptide region comprising or consisting of SEQ ID NO: 8; or a variant δ chain extracellular region less the connecting peptide region which has at least 88% sequence identity to the δ chain extracellular region less the connecting peptide region of SEQ ID NO: 8 (e.g. at least 89%, 90%, 91%, 92%, 93%, 94%, 95%, 96%, 97%, 98% or 99% sequence identity therewith).

An example of a variant of SEQ ID NO: 8 is SEQ ID NO: 14 and SEQ ID NO: 18.

In an embodiment, the tumour-specific T-cell receptor (TCR) or tumour-specific binding fragment of a TCR comprises a δ chain extracellular region less the connecting peptide region comprising or consisting of SEQ ID NO: 14; or a variant δ chain extracellular region less the connecting peptide region which has at least 88% sequence identity to the δ chain extracellular region less the connecting peptide region of SEQ ID NO: 14 (e.g. at least 89%, 90%, 91%, 92%, 93%, 94%, 95%, 96%, 97%, 98% or 99% sequence identity therewith).

An example of a variant of SEQ ID NO: 14 is SEQ ID NO: 8 and SEQ ID NO: 18.

In an embodiment, the tumour-specific T-cell receptor (TCR) or tumour-specific binding fragment of a TCR comprises a δ chain extracellular region less the connecting peptide region comprising or consisting of SEQ ID NO: 18; or a variant δ chain extracellular region less the connecting peptide region which has at least 88% sequence identity to the δ chain extracellular region less the connecting peptide region of SEQ ID NO: 18 (e.g. at least 89%, 90%, 91%, 92%, 93%, 94%, 95%, 96%, 97%, 98% or 99% sequence identity therewith).

An example of a variant of SEQ ID NO: 18 is SEQ ID NO: 8 and SEQ ID NO: 14.

In an embodiment, the tumour-specific T-cell receptor (TCR) or tumour-specific binding fragment of a TCR comprises a δ chain extracellular region less the connecting peptide region comprising or consisting of SEQ ID NO:8.

In an embodiment, the tumour-specific T-cell receptor (TCR) or tumour-specific binding fragment of a TCR comprises a δ chain extracellular region less the connecting peptide region comprising or consisting of SEQ ID NO: 14.

In an embodiment, the tumour-specific T-cell receptor (TCR) or tumour-specific binding fragment of a TCR comprises a δ chain extracellular region less the connecting peptide region comprising or consisting of SEQ ID NO: 18.

In yet a further preferred embodiment of the invention said TCR comprises said aforementioned TCR γ chain and said aforementioned TCR δ chain.

Suitably, the TCR of the invention comprises:
(a) a γ chain comprising a CDR3 sequence of SEQ ID NO: 1 or a variant CDR3 which has at least 88% sequence identity to the CDR3 of SEQ ID NO: 1 (e.g. at least 89%, 90%, 91%, 92%, 93%, 94%, 95%, 96%, 97%, 98% or 99% sequence identity therewith), a CDR1 sequence of SEQ ID NO: 3 and CDR2 sequence of SEQ ID NO: 4 and a δ chain comprising a CDR3 sequence of SEQ ID NO: 2 or a variant CDR which has at least 88% sequence identity to the CDR of SEQ ID NO: 1 (e.g. at least 89%, 90%, 91%, 92%, 93%, 94%, 95%, 96%, 97%, 98% or 99% sequence identity therewith), a CDR1 sequence of SEQ ID NO: 5 and CDR2 sequence of SEQ ID NO: 6; or
(b) a γ chain extracellular region less the connecting peptide region comprising or consisting of SEQ ID NO:7 or a variant γ chain extracellular region less the connecting peptide region which has at least 88% sequence identity to the γ chain extracellular region less the connecting peptide region of SEQ ID NO: 7 and a δ chain extracellular region less the connecting peptide region comprising SEQ ID NO: 8 or a variant δ chain extracellular region less the connecting peptide region which has at least 88% sequence identity to the δ chain extracellular region less the connecting peptide of SEQ ID NO: 8 or a δ chain extracellular region less the connecting peptide region comprising SEQ ID NO: 14 or a variant δ chain extracellular region less the connecting peptide region which has at least 88% sequence identity to the δ chain extracellular region less the connecting peptide region of SEQ ID NO: 14; or
(c) a γ chain extracellular region less the connecting peptide region comprising or consisting of SEQ ID NO:17 or a variant γ chain extracellular region less the connecting peptide region which has at least 88% sequence identity to the γ chain extracellular region less the connecting peptide of SEQ ID NO: 17 and a δ chain extracellular region less the connecting peptide region comprising SEQ ID NO: 18 or a variant δ chain extracellular region less the connecting peptide region which has at least 88% sequence identity to the δ chain extracellular region less the connecting peptide of SEQ ID NO: 18; or
(d) a γ chain comprising or consisting of SEQ ID NO: 15 minus the N-terminal leader sequence (residues 1-18) or a variant γ chain which has at least 88% sequence identity to they chain of SEQ ID NO: 15 minus the N-terminal leader sequence and a δ chain comprising or consisting of SEQ ID NO: 16 minus the N-terminal leader sequence (residues 1-20) or a variant δ chain which has at least 88% sequence identity to the δ chain of SEQ ID NO: 16 minus the N-terminal leader sequence.

In an embodiment, the TCR or tumour-specific binding fragment thereof is specific for at least one SCNNA1 gene product isoforms. Suitably, the TCR or tumour-specific binding fragment thereof binds to at least one of the SCNNA1 gene product isoforms encoded by the amino acid sequence of SEQ ID NOs: 29 to 34. More suitably, the TCR or tumour specific binding fragment thereof binds to the extracellular domain of said isoform.

In yet another preferred embodiment of the invention said TCR is part of a chimeric receptor having the functionality described herein.

TCR polypeptide sequences of the invention can be obtained and manipulated using the techniques disclosed for example in Green and Sambrook 2012 Molecular Cloning: A Laboratory Manual 4th Edition Cold Spring Harbour Laboratory Press.

### Sequence Comparisons

For the purposes of comparing two closely-related polypeptide sequences, the “% sequence identity” between a first sequence and a second sequence may be calculated. Polypeptide sequences are said to be the same as or identical to other polypeptide sequences, if they share 100% sequence identity over their entire length. Residues in sequences are numbered from left to right, i.e. from N- to C-terminus for polypeptides. The terms “identical” or percentage “identity”, in the context of two or more polypeptide sequences, refer to two or more sequences or sub-sequences that are the same or have a specified percentage of amino acid residues that are the same (i.e. at least 88%, 89%, 90%, 91%, 92%, 93%, 94%, 95%, 96%, 97%, 98% or 99% A sequence identity over a specified region), when compared and aligned for maximum correspondence over a comparison window. Suitably, the comparison is performed over a window corresponding to the entire length of the reference sequence.

For sequence comparison, one sequence acts as the reference sequence, to which the test sequences are compared. When using a sequence comparison algorithm, test and reference sequences are entered into a computer, subsequent coordinates are designated, if necessary, and sequence algorithm program parameters are designated. Default program parameters can be used, or alternative parameters can be designated. The sequence comparison algorithm then calculates the percentage sequence identities for the test sequences relative to the reference sequence, based on the program parameters.

A “comparison window”, as used herein, refers to a segment in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, 1981, Adv. Appl. Math. 2:482, by the homology alignment algorithm of Needleman & Wunsch, 1970, J. Mol. Biol. 48:443, by the search for similarity method of Pearson & Lipman, 1988, Proc. Nat'l. Acad. Sci. USA 85:2444, by computerised implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

An example of an algorithm that is suitable for determining percent sequence identity and sequence similarity are the BLAST and BLAST 2.0 algorithms, which are described in Altschul et al., 1977, Nuc. Acids Res. 25:3389-3402 and Altschul et al., 1990, J. Mol. Biol. 215:403-410, respectively. Software for performing BLAST analyses is publicly available through the National Center for Biotechnology Information (website at www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighbourhood word score threshold (Altschul et al., supra). These initial neighbourhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. For amino acid sequences, the BLASTP program uses as defaults a wordlength of 3, and expectation (E) of 10, and the BLOSUM62 scoring matrix (see Henikoff & Henikoff, 1989, Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin & Altschul, 1993, Proc. Nat'l. Acad. Sci. USA 90:5873-5787). One measure of similarity provided by the BLAST algorithm is the smallest sum probability (P(N)), which provides an indication of the probability by which a match between two nucleotide or amino acid sequences would occur by chance.

A “difference” between sequences refers to an insertion, deletion or substitution of a single residue in a position of the second sequence, compared to the first sequence. Two sequences can contain one, two or more such differences. Insertions, deletions or substitutions in a second sequence which is otherwise identical (100% sequence identity) to a first sequence result in reduced % sequence identity. For example, if the identical sequences are 9 residues long, one substitution in the second sequence results in a sequence identity of 88.9%. If the identical sequences are 17 amino acid residues long, two substitutions in the second sequence results in a sequence identity of 88.2%.

Alternatively, for the purposes of comparing a first, reference sequence to a second, comparison sequence, the number of additions, substitutions and/or deletions made to the first sequence to produce the second sequence may be ascertained. An addition is the addition of one residue into the first sequence (including addition at either terminus of the first sequence). A substitution is the substitution of one residue in the first sequence with one different residue. A deletion is the deletion of one residue from the first sequence (including deletion at either terminus of the first sequence).

### Sequence Variants

The term “amino acid” refers to any one of the naturally occurring amino acids, as well as amino acid analogues and amino acid mimetics that function in a manner which is similar to the naturally occurring amino acids. Naturally occurring amino acids are those 20 L-amino acids encoded by the genetic code, as well as those amino acids that are later modified, e.g., hydroxyproline, γ-carboxyglutamate, and O-phosphoserine. The term “amino acid analogue” refers to a compound that has the same basic chemical structure as a naturally occurring amino acid, i.e., an α carbon that is bound to a hydrogen, a carboxyl group, an amino group, and an R group but has a modified R group or a modified peptide backbone as compared with a natural amino acid. Examples include homoserine, methionine sulfoxide, methionine methyl sulfonium and norleucine. Amino acid mimetics refers to chemical compounds that have a structure that is different from the general chemical structure of an amino acid, but that functions in a manner similar to a naturally occurring amino acid. Suitably an amino acid is a naturally occurring amino acid or an amino acid analogue, especially a naturally occurring amino acid and in particular one of those 20 L-amino acids encoded by the genetic code.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

In an embodiment, the amino acid sequence of the TCR or tumour-specific binding fragment thereof is artificial. Without limitation, and as discussed further herein, the TCR or tumour-specific binding fragment thereof may comprise at least one mutation to remove a cysteine residue by replacement with another residue and/or to introduce a cysteine residue by replacement of another residue with cysteine.

In an embodiment, at least one amino acid is substituted, added or deleted relative to the wildtype sequence.

In an embodiment, the at least one amino acid is (are) located in a framework region, a CDR or a constant region, particularly in a framework region or a constant region, especially in a constant region. Thus, particularly, any and all additions, substitutions and deletions of amino acids are in a framework region or a constant region, particularly in a constant region.

In an embodiment, in respect of variation in the sequences of SEQ ID NOs: 7-8 and 14-18, the at least one amino acid is not located in any CDR.

Variations in sequence can be in the form of additions, substitutions and deletions, especially substitutions. For example, additions can be at the N and/or C termini of sequences and deletions can be at the N and/or C termini of sequences. There may, for example, be 1 addition, substitution or deletion in the sequence of SEQ ID NO: 1. There may, for example, be 1 or 2 (particularly 1) additions, substitutions and deletions in the sequence of SEQ ID NO: 2. There may, for example, be up to 24 e.g. up to 20 e.g. up to 15 e.g. up to 10 e.g. up to 5 e.g. 4, 3, 2 or 1) additions, substitutions and deletions in the sequences of SEQ ID NOs: 7-8 and 14-18.

Substitutions are suitably conservative substitutions. The following eight groups each contain amino acids that are typically conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M)
(see, e.g., Creighton, Proteins 1984).

Suitably, sequence variations do not significantly adversely affect the ability of the TCR or fragment thereof to bind to its target epitope on the tumour, for example its binding affinity of the variant is 75% or more e.g. 80% or more e.g. 85% or more e.g. 90% or more e.g. 95% or more e.g. 98% or more e.g. 99% or more of that of the TCR that in soluble form has γ and δ chains of SEQ ID NOs: 7 and 8 respectively.

The following variants formed of sequence mutations are contemplated in particular:

Particularly useful mutations are mutations made to one or both chains (i.e. one or both of the γ and δ chains) of a TCR of the invention to reduce the likelihood that said chain or chains will pair with a chain or chains of the TCR naturally produced by a T-cell. For example, mutations may be made in the constant region of the extracellular region to promote pairing of the recombinant chains and reduce pairing with an endogenous TCR. One such example is the introduction into the constant region of the extracellular region of the γ and δ chains of a cysteine (C) residue such that the TCR forms a disulphide bridge not present in the naturally produced TCR. Suitably the C residue replaces a G or A residue. For example, position 176 of the extracellular region of the γ chain within the constant region (naturally G—see SEQ ID NO: 7) is mutated to C (see SEQ ID NO: 17). For example, position 163 of the extracellular region of the δ chain within the constant region (naturally A—see SEQ ID NO: 14) is mutated to C (see SEQ ID NO: 18). Preferably both of these changes are made.

Instead or as well, the constant region (all or substantially all thereof, such as at least 95% e.g. at least 99% of its residues) of one or preferably both chains of the TCR may be replaced by the corresponding sequence of the constant region of a murine TCR. Such a replacement (known as “murinisation”) may also promote pairing of recombinant chains and reduce pairing with an endogenous TCR. Sequence variants of the murine constant region may also employed in a similar manner to that described for variants of the TCR sequences, including sequences having 90%, 91%, 92%, 93%, 94%, 95%, 96%, 97%, 98% or 99% sequence identity therewith. For example, 1 or 2 residues may be mutated.

The sequence of an example murine constant region sequence of a TCR γ chain is provided below. The X at the beginning of the sequence in position 1 may be any naturally occurring amino acid.

(SEQ ID NO: 19)
XKRLDADISPKPTIFLPSVAETNLHKTGTYLCLLEKFFPDVIRVYWKEKDG
NTILDSQEGDTLKTNDTYMKFSWLTVPERAMGKEHRCIVKHENNKGGADQE
IFFPSIKKVAVSTKPTTCWQDKNDVLQLQFTITSAYYTYLLLLLKSVIYLA
IISFSLLRRTSVCGNEKKS.

According to a suitable variant sequence of SEQ ID NO: 19, residue 60 (glycine, underlined) is mutated to C.

The sequence of an example murine constant region sequence of a TCR δ chain sequence is provided below. The X at the beginning of the sequence in position 1 may be any naturally occurring amino acid.

(SEQ ID NO: 20)
XSQPPAKPSVFIMKNGTNVACLVKDFYPKEVTISLRSSKKIVEFDPAIVIS
PSGKYSAVKLGQYGDSNSVTCSVQHNSETVHSTDFEPYANSFNNEKLPEPE
NDTQISEPCYGPRVTVHTEKVNMMSLTVLGLRLLFAKTIAINFLLTVKLF
F.

According to a suitable variant sequence of SEQ ID NO: 20, residue 47 (alanine, underlined) is mutated to C.

The mutations G60C in SEQ ID NO: 19 and A47CC in SEQ ID NO: 20 lead to the formulation of a new disulphide bridge in the heterodimer TCR which increases the stability of the TCR.

Further useful mutations that may be made include mutations intended to increase the stability of a domain and/or promote correct folding, especially the extracellular domain of a soluble TCR. For example, residue 117 in the extracellular region of the δ chain (naturally R—see SEQ ID NO: 14) may be replaced with N (see SEQ ID NO: 8). Other possible mutations include the removal of native cysteine residues (particularly in the instance when new cysteine residues are introduced as mentioned above). Thus, existing C residues may be removed in the extracellular region (particularly within the constant region thereof) of either or both of the chains. For example, an existing C residue in the extracellular region of the δ chain within the constant region may be replaced with A, S or T, especially A. An existing C residue in the extracellular region of the γ chain within the constant region may also (or instead) be replaced with A, S or T, especially A.

Sequences for expression in a bacterial host may be provided with an initial M residue.

### Sequence Fragments

Fragments of the TCR of the invention are provided which retain the function of tumour-specific binding.

Suitably, fragments substantially retain the ability of the TCR or fragment thereof to bind to its target epitope on the tumour, for example the binding affinity of the fragment is 75% or more e.g. 80% or more e.g. 85% or more e.g. 90% or more e.g. 95% or more e.g. 98% or more e.g. 99% or more of that of the TCR that in soluble form has γ and δ chains of SEQ ID NOs: 7 and 8 respectively.

In an embodiment, said TCR is a soluble TCR, or sTCR, and so lacks the transmembrane and, ideally also, intracellular domain. Thus, in an embodiment the TCR is a soluble form of a TCR which lacks the transmembrane and the intracellular domains i.e. it consists only of the extracellular domain. In another embodiment the TCR is a soluble form of a TCR which lacks the connecting peptide region of the extracellular domain and lacks the transmembrane and the intracellular domains i.e. it consists only of the extracellular domain less the connecting peptide region.

In respect of specific sequences disclosed herein relating to the extracellular domain less the connecting peptide region: the complete extracellular domain of the γ chain may be formed by adding residues 245-276 of SEQ ID NO: 15 to its C terminus; the complete extracellular domain of the δ chain may be formed by adding residues 230-265 of SEQ ID NO: 16 to its C terminus.

In an embodiment, a single chain of the TCR is provided e.g. the γ chain or the δ chain. Thus, an example γ chain sequence comprises or consists of SEQ ID NO: 7 or a sequence having at least 88% sequence identity therewith. An example δ chain sequence comprises or consists of SEQ ID NO: 8 or a sequence having at least 88% sequence identity therewith.

A further exemplary fragment which is a fragment of a γ chain comprises SEQ ID NOs: 1, 3 and 5, for example, comprise residues 27-107 of SEQ ID NO: 7. A further exemplary fragment which is a fragment of a δ chain comprises SEQ ID NOs: 2, 4 and 6, for example, comprises residues 27-106 of SEQ ID NO: 8.

A further exemplary fragment comprises a fragment of a γ chain which comprises SEQ ID NOs: 1, 3 and 5, for example, comprises residues 27-107 of SEQ ID NO: 7 and comprises a fragment of a δ chain which comprises SEQ ID NOs: 2, 4 and 6, for example, comprises residues 27-106 of SEQ ID NO: 8.

In an embodiment, said TCR comprises an extracellular domain, a transmembrane domain and an intracellular domain. Hence each chain of the TCR comprises an extracellular region, a transmembrane region and an intracellular region.

Variants of fragments may also be contemplated (see above discussion of sequence variants).

### Polynucleotides and Vectors

According to a further aspect of the invention there is provided a polynucleotide encoding the TCR or tumour-specific binding fragment of the invention.

In an embodiment, a polynucleotide encoding the γ chain of the TCR has the sequence:

(SEQ ID NO: 21)
ATGAGGTGGGCCCTGGCCGTGCTCCTGGCTTTTCTCAGCCCTGCCTCCCAG
AAGAGCTCCAACCTGGAGGGAAGGACCAAGTCCGTGACCAGACAGACAGGC
AGCAGCGCCGAGATCACCTGTGATCTGACCGTGACCAATACCTTTTACATC
CATTGGTACCTGCATCAGGAGGGCAAGGCCCCTCAAAGGCTGCTCTACTAC
GATGTCTCCACCGCCAGAGATGTGCTGGAATCCGGACTGAGCCCCGGCAAA
TATTACACCCATACCCCCAGGAGGTGGTCCTGGATCCTGAGACTGCAGAAT
CTGATCGAGAATGACAGCGGCGTGTACTACTGCGCCACCTGGGACAGAAGG
GATTACAAGAAGCTGTTCGGCAGCGGCACAACCCTGGTCGTGACCGACAAG
CAGCTGGACGCCGATGTGAGCCCTAAGCCCACAATCTTTCTGCCCTCCATC
GCTGAGACCAAACTGCAGAAGGCCGGCACATATCTCTGTCTCCTGGAGAAG
TTTTTTCCCGACGTGATCAAGATCCATTGGCAGGAGAAGAAGAGCAACACC
ATTCTCGGCAGCCAGGAAGGCAATACCATGAAAACAAATGACACATACATG
AAGTTTTCCTGGCTGACAGTCCCCGAGAAAAGCCTCGACAAGGAACACAGG
TGCATCGTCAGGCACGAGAACAATAAGAACGGCGTGGACCAAGAGATCATT
TTTCCTCCTATCAAAACAGACGTCATCACAATGGATCCCAAGGACAACTGC
AGCAAGGATGCCAATGACACCCTGCTCCTGCAGCTGACCAACACCAGCGCC
TACTACATGTACCTGCTGCTGCTCCTGAAGTCCGTGGTGTACTTCGCTATC
ATCACATGCTGCCTGCTGAGAAGAACCGCCTTCTGTTGCAACGGAGAGAAG
TCC

SEQ ID NO: 21 is the native DNA sequence of the native γ chain of the TCR of clone SW.3G1 according to the invention.

The sequence of SEQ ID NO: 21 does not include a stop codon. A suitable stop codon e.g. TAA will typically be included at the C terminus in a construct including this sequence unless a run through translation of the sequence as part of a fusion is required.

In an embodiment, a polynucleotide encoding the δ chain of the TCR has the sequence:

(SEQ ID NO: 22)
ATGCTGTTCTCCAGCCTGCTGTGTGTGTTTGTCGCCTTCAGCTATAGCGGC
TCCAGCGTGGCCCAGAAGGTGACCCAGGCCCAGTCCAGCGTGAGCATGCCC
GTGAGGAAGGCCGTCACCCTGAACTGTCTGTACGAGACCAGCTGGTGGAGC
TACTACATCTTCTGGTACAAGCAGCTGCCCAGCAAGGAGATGATCTTTCTG
ATCAGGCAGGGCTCCGATGAACAGAATGCCAAGAGCGGCAGGTACTCCGTG
AACTTCAAGAAGGCCGCCAAAAGCGTCGCCCTGACCATCTCCGCCCTGCAG
TTAGAAGACTCCGCCAAGTACTTTTGTGCCCTGGGAGTGCTGCCTACCGTG
ACCGGAGGCGGCCTGATCTTTGGCAAAGGAACCAGGGTCACCGTGGAGCCT
AGATCCCAACCCCACACCAAACCCAGCGTGTTCGTGATGAAAAACGGCACC
AACGTGGCCTGTCTCGTCAAGGAATTTTACCCCAAAGACATCAGGATCAAC
CTCGTGTCCTCCAAGAAGATCACCGAGTTCGACCCTGCCATCGTGATCTCC
CCTAGCGGAAAGTACAACGCCGTGAAACTGGGAAAGTACGAGGATTCCAAC
TCCGTGACCTGCTCCGTCCAACATGACAACAAGACCGTGCACAGCACCGAC
TTCGAGGTGAAGACAGATAGCACCGATCACGTGAAGCCCAAGGAAACAGAG
AACACCAAGCAGCCTAGCAAGTCCTGCCACAAGCCCAAGGCCATCGTCCAC
ACCGAGAAAGTCAACATGATGAGCCTGACCGTGCTGGGCCTGAGGATGCTG
TTTGCCAAGACCGTCGCCGTGAATTTCCTGCTGACCGCCAAACTCTTCTTC
CTG

SEQ ID NO: 22 is the native DNA sequence of the native δ chain of the TCR of clone SW.3G1 according to the invention.

The sequence of SEQ ID NO: 22 does not include a stop codon. A suitable stop codon e.g. TAA will typically be included at the C terminus in a construct including this sequence unless a run through translation of the sequence as part of a fusion is required.

In an embodiment, such a polynucleotide may be a chimeric polynucleotide comprising a gene encoding the TCR or tumour-specific binding fragment and a heterologous promoter and/or other transcription control element such as a terminating signal operably linked thereto.

Since a complete TCR comprises a γ chain and a δ chain, two polynucleotides each encoding a chain of the TCR may be provided or a polynucleotide encoding both chains of the TCR may be provided. Yet further, the two chains of the TCR may be linked by a cleavable peptide linker (e.g. a linker that cleaves in a T-cell; including a “self-cleaving” viral 2A sequence) and the polynucleotide may encode both chains of the TCR and the linker (see SEQ ID NO: 21 discussed below).

The terms “nucleic acid” and “polynucleotide” are used interchangeably herein and refer to a polymeric macromolecule made from nucleotide monomers particularly deoxyribonucleotide or ribonucleotide monomers. The term encompasses polynucleotides containing known nucleotide analogues or modified backbone residues or linkages, which are naturally occurring and non-naturally occurring, which have similar properties as the reference polynucleotide, and which are intended to be metabolized in a manner similar to the reference nucleotides or are intended to have extended half-life in the system. Examples of such analogues include, without limitation, phosphorothioates, phosphoramidates, methyl phosphonates, chiral-methyl phosphonates, 2-O-methyl ribonucleotides, peptide-nucleic acids (PNAs). Suitably the term “polynucleotide” refers to naturally occurring polymers of deoxyribonucleotide or ribonucleotide monomers. Suitably the polynucleotides of the invention are recombinant. Recombinant means that the polynucleotide is the product of at least one of cloning, restriction or ligation steps, or other procedures that result in a polynucleotide that is distinct from a polynucleotide found in nature (e.g., in the case of cDNA). In an embodiment the polynucleotide of the invention is an artificial polynucleotide sequence (e.g., a cDNA sequence or polynucleotide sequence with non-naturally occurring codon usage). In one embodiment, the polynucleotides of the invention are DNA. Alternatively, the polynucleotides of the invention are RNA.

DNA (deoxyribonucleic acid) and RNA (ribonucleic acid) refer to polynucleotides having a backbone of sugar moieties which are deoxyribosyl and ribosyl moieties respectively. The sugar moieties may be linked to bases which are the 4 natural bases (adenine (A), guanine (G), cytosine (C) and thymine (T) in DNA and adenine (A), guanine (G), cytosine (C) and uracil (U) in RNA). As used herein, a “corresponding RNA” is an RNA having the same sequence as a reference DNA but for the substitution of thymine (T) in the DNA with uracil (U) in the RNA. The sugar moieties may also be linked to unnatural bases such as inosine, xanthosine, 7-methylguanosine, dihydrouridine and 5-methylcytidine. Natural phosphodiester linkages between sugar (deoxyribosyl/ribosyl) moieties may optionally be replaced with phosphorothioates linkages. Suitably polynucleotides of the invention consist of the natural bases attached to a deoxyribosyl or ribosyl sugar backbone with phosphodiester linkages between the sugar moieties.

In an embodiment the polynucleotide of the invention is a DNA, including single- or double-stranded DNA and straight-chain or circular DNA (i.e. plasmid DNA).

Due to the degeneracy of the genetic code, a large number of different, but functionally identical polynucleotides can encode any given polypeptide. For instance, the codons GCA, GCC, GCG and GCU all encode the amino acid alanine. Thus, at every position where an alanine is specified by a codon, the codon can be altered to any of the corresponding codons described without altering the encoded polypeptide. Such polynucleotide variations lead to “silent” (sometimes referred to as “degenerate” or “synonymous”) variants, which are one species of conservatively modified variations. Every polynucleotide sequence disclosed herein which encodes a polypeptide also enables every possible silent variation of the polynucleotide. One of skill will recognise that each codon in a polynucleotide (except AUG, which is ordinarily the only codon for methionine, and UGG, which is ordinarily the only codon for tryptophan) can be modified to yield a functionally identical molecule. Accordingly, each silent variation of a polynucleotide that encodes a polypeptide is implicit in each described sequence and is provided as an aspect of the invention.

Degenerate codon substitutions may also be achieved by generating sequences in which the third position of one or more selected (or all) codons is substituted with mixed-base and/or deoxyinosine residues (Batzer et al., 1991, Nucleic Acid Res. 19:5081; Ohtsuka et al., 1985, J. Biol. Chem. 260:2605-2608; Rossolini et al., 1994, Mol. Cell. Probes 8:91-98).

Codons of the polynucleotide sequences of the invention may be altered in order that sequence variants of the TCR are expressed as discussed above. In an embodiment, up to 5 codons are altered e.g. one, two or three e.g. one or two e.g. one codons are altered such that a different amino acid is encoded where the codon alteration occurs. Codon alterations may involve the alteration of one, two or three bases in the polynucleotide according to the amino acid alteration to be achieved. Suitably, codons encoding residues of the CDRs are not altered.

In an embodiment, the polynucleotides of the invention are codon optimised for expression in a human host cell, particularly, a T-cell.

According to a yet further aspect of the invention there is provided a vector encoding said TCR or tumour-specific binding fragment of the invention. Specifically, there is provided a vector for delivery of a polynucleotide to cells (particularly T-cells) comprising a polynucleotide encoding the TCR or tumour-specific binding fragment of the invention.

As noted above, since a complete TCR comprises a γ chain and a δ chain, two vectors each comprising a polynucleotide encoding a chain of the TCR may be provided or a vector comprising a polynucleotide encoding both chains of the TCR may be provided. Yet further, the two chains of the TCR may be linked by a cleavable peptide linker (e.g. a linker that cleaves in a T-cell) and the vector may comprise a polynucleotide which encodes both chains of the TCR and the linker.

The or each vector should suitably comprise such elements as are necessary for permitting transcription of a translationally active RNA molecule in the host cell, such as a promoter and/or other transcription control elements such as an internal ribosome entry site (IRES) or a termination signal. A “translationally active RNA molecule” is an RNA molecule capable of being translated into a protein by the host cell's translation apparatus.

Example promoters to drive transcription of the TCR chains include constitutive promoters such as the cytomegalovirus (CMV) promoter and elongation factor 1α (EF1α) promoter.

The vector may be, for example, a viral vector such as a lentiviral vector. Other examples of viral vectors include vectors derived from γ-retrovirus, adenovirus, adeno-associated virus (AAV), alphavirus, herpes virus, arenavirus, measles virus, poxvirus or rhabdovirus. DNA molecules, for example transposons, may also be suitable vectors to transduce T cells with TCR genes.

A suitable polynucleotide of the invention may encode a TCR which comprises:
(a) a γ chain comprising a CDR3 sequence of SEQ ID NO: 1 or a variant CDR3 which has at least 88% sequence identity to the CDR3 of SEQ ID NO: 1 (e.g. at least 89%, 90%, 91%, 92%, 93%, 94%, 95%, 96%, 97%, 98% or 99% sequence identity therewith), a CDR1 sequence of SEQ ID NO: 3 and CDR2 sequence of SEQ ID NO: 4 and a δ chain comprising a CDR3 sequence of SEQ ID NO: 2 or a variant CDR3 which has at least 88% sequence identity to the CDR3 of SEQ ID NO: 2 (e.g. at least 89%, 90%, 91%, 92%, 93%, 94%, 95%, 96%, 97%, 98% or 99% sequence identity therewith), a CDR1 sequence of SEQ ID NO: 5 and CDR2 sequence of SEQ ID NO: 6; or
(b) a γ chain extracellular region less the connecting peptide comprising or consisting of SEQ ID NO:7 or a variant γ chain extracellular region less the connecting peptide which has at least 88% sequence identity to the γ chain extracellular region less the connecting peptide of SEQ ID NO: 7 and a δ chain extracellular region less the connecting peptide comprising SEQ ID NO:8 or a variant δ chain extracellular region less the connecting peptide which has at least 88% sequence identity to the δ chain extracellular region less the connecting peptide of SEQ ID NO: 8; or δ chain extracellular region less the connecting peptide comprising SEQ ID NO: 14 or a variant δ chain extracellular region less the connecting peptide which has at least 88% sequence identity to the δ chain extracellular region less the connecting peptide of SEQ ID NO: 14; or
(c) a γ chain extracellular region less the connecting peptide comprising or consisting of SEQ ID NO: 17 or a variant γ chain extracellular region less the connecting peptide which has at least 88% sequence identity to the γ chain extracellular region less the connecting peptide of SEQ ID NO: 17 and a δ chain extracellular region less the connecting peptide comprising SEQ ID NO: 18 or a variant δ chain extracellular region less the connecting peptide which has at least 88% sequence identity to the δ chain extracellular region less the connecting peptide of SEQ ID NO: 18; or
(d) a γ chain comprising or consisting of SEQ ID NO: 15 minus the N-terminal leader sequence (residues 1-18) or a variant γ chain which has at least 88% sequence identity to the γ chain of SEQ ID NO: 15 minus the N-terminal leader sequence and a δ chain comprising or consisting of SEQ ID NO: 16 minus the N-terminal leader sequence (residues 1-20) or a variant δ chain which has at least 88% sequence identity to the δ chain of SEQ ID NO: 16 minus the N-terminal leader sequence.

Said polynucleotide may suitably encode an immature TCR which comprises an N-terminal leader sequence (residues 1-18 of SEQ ID NO: 15 and residues 1-20 of SEQ ID NO: 16) which N-terminal leader sequence is removed by cellular peptidases (such as signal peptidase) to produce the mature form.

A suitable vector of the invention comprises the aforementioned polynucleotide and is, for example, a viral vector as disclosed above such as a lentiviral vector.

### Production of TCR Receptors

TCRs of the invention and the derivatives described herein (bispecifics etc.) can be obtained and manipulated using the techniques disclosed for example in Green and Sambrook 2012 Molecular Cloning: A Laboratory Manual 4th Edition Cold Spring Harbour Laboratory Press. In particular, artificial gene synthesis may be used to produce polynucleotides (Nambiar et al., 1984, Science, 223:1299-1301, Sakamar and Khorana, 1988, Nucl. Acids Res., 14:6361-6372, Wells et al., 1985, Gene, 34:315-323 and Grundstrom et al., 1985, Nucl. Acids Res., 13:3305-3316) followed by expression in a suitable organism to produce polypeptides. A gene encoding a polypeptide of the invention can be synthetically produced by, for example, solid-phase DNA synthesis. Entire genes may be synthesized de novo, without the need for precursor template DNA. To obtain the desired oligonucleotide, the building blocks are sequentially coupled to the growing oligonucleotide chain in the order required by the sequence of the product. Upon the completion of the chain assembly, the product is released from the solid phase to solution, deprotected, and collected. Products can be isolated by high-performance liquid chromatography (HPLC) to obtain the desired oligonucleotides in high purity (Verma and Eckstein, 1998, Annu. Rev. Biochem. 67:99-134). These relatively short segments are readily assembled by using a variety of gene amplification methods (Methods Mol Biol., 2012; 834:93-109) into longer DNA molecules, suitable for use in innumerable recombinant DNA-based expression systems. In the context of this invention one skilled in the art would understand that the polynucleotide sequences encoding the TCRs and fragments thereof described in this invention could be readily used in a variety of protein production systems, including, for example, viral vectors.

For the purposes of production of polypeptides of the invention in a microbiological host (e.g., bacterial or fungal), polynucleotides of the invention will comprise suitable regulatory and control sequences (including promoters, termination signals etc) and sequences to promote polypeptide secretion suitable for protein production in the host. Similarly, polypeptides of the invention could be produced by transducing cultures of eukaryotic cells (e.g., Chinese hamster ovary cells or drosophila S2 cells) with polynucleotides of the invention which have been combined with suitable regulatory and control sequences (including promoters, termination signals etc) and sequences to promote polypeptide secretion suitable for protein production in these cells.

Improved isolation of the polypeptides of the invention produced by recombinant means may optionally be facilitated through the addition of a purification tag at one end of the polypeptide. An example purification tag is a stretch of histidine residues (e.g. 6-10 His residues), commonly known as a His-tag. Other example purification tags include a MYC tag with amino acid sequence EQKLISEEDL (SEQ ID NO: 26), a FLAG tag with sequence DYKDDDDK (SEQ ID NO: 27) or an HA tag with sequence YPYDVPDYA (SEQ ID NO: 28).

The polypeptides of the invention may be produced ex vivo in T-cells as discussed below.

### T-Cells and T-Cell Clones

According to a further aspect of the invention there is provided a T-cell expressing said TCR or tumour-specific binding fragment of the invention, ideally, in either a soluble or membrane compatible form i.e. having a transmembrane region and intracellular region.

According to a yet further aspect of the invention there is provided a T-cell clone expressing said TCR or tumour-specific binding fragment of the invention, ideally, in either a soluble or membrane compatible form i.e. having a transmembrane region and intracellular region. Preferably said clone is a SW.3G1 clone as described herein.

According to an aspect of the invention there is provided an engineered T-cell or T-cell clone which expresses a TCR of the invention, in particular, it expresses a TCR which comprises:
(a) a γ chain comprising a CDR3 sequence of SEQ ID NO: 1 or a variant CDR3 which has at least 88% sequence identity to the CDR of SEQ ID NO: 1 (e.g. at least 89%, 90%, 91%, 92%, 93%, 94%, 95%, 96%, 97%, 98% or 99% sequence identity therewith), a CDR1 sequence of SEQ ID NO: 3 and CDR2 sequence of SEQ ID NO: 4 and a δ chain comprising a CDR3 sequence of SEQ ID NO: 2 or a variant CDR3 which has at least 88% sequence identity to the CDR of SEQ ID NO: 1 (e.g. at least 89%, 90%, 91%, 92%, 93%, 94%, 95%, 96%, 97%, 98% or 99% sequence identity therewith), a CDR1 sequence of SEQ ID NO: 5 and CDR2 sequence of SEQ ID NO: 6; or
(b) a γ chain extracellular region less the connecting peptide comprising or consisting of SEQ ID NO:7 or a variant γ chain extracellular region less the connecting peptide which has at least 88% sequence identity to the γ chain extracellular region less the connecting peptide of SEQ ID NO: 7 and a δ chain extracellular region less the connecting peptide comprising SEQ ID NO:8 or a variant δ chain extracellular region less the connecting peptide which has at least 88% sequence identity to the δ chain extracellular region less the connecting peptide of SEQ ID NO: 8 or a δ chain extracellular region less the connecting peptide comprising SEQ ID NO: 14 or a variant δ chain extracellular region less the connecting peptide which has at least 88% sequence identity to the δ chain extracellular region less the connecting peptide of SEQ ID NO: 14; or
(c) a γ chain extracellular region less the connecting peptide comprising or consisting of SEQ ID NO:17 or a variant γ chain extracellular region less the connecting peptide which has at least 88% sequence identity to the γ chain extracellular region less the connecting peptide of SEQ ID NO: 17 and a δ chain extracellular region less the connecting peptide comprising SEQ ID NO: 18 or a variant δ chain extracellular region less the connecting peptide which has at least 88% sequence identity to the δ chain extracellular region less the connecting peptide of SEQ ID NO: 18; or
(d) a γ chain comprising or consisting of SEQ ID NO: 15 minus the N-terminal leader sequence (residues 1-18) or a variant γ chain which has at least 88% sequence identity to the γ chain of SEQ ID NO: 15 minus the N-terminal leader sequence and a δ chain comprising or consisting of SEQ ID NO: 16 minus the N-terminal leader sequence (residues 1-20) or a variant δ chain which has at least 88% sequence identity to the δ chain of SEQ ID NO: 16 minus the N-terminal leader sequence.

Suitably the T-cell is a CD8+ T-cell.

In an embodiment the T-cell or T-cell clone is engineered. For example, the invention provides a T cell or T-cell clone wherein the cell or cells of the clone are transduced with a heterologous polynucleotide encoding the TCR or tumour specific binding fragment of the invention or by a vector of the invention.

In an embodiment, the T-cell comprises a vector comprising a polynucleotide encoding the TCR of the invention and the T-cell expresses the TCR of the invention.

According to an aspect of the invention there is provided an ex vivo process comprising (i) obtaining T-cells from a patient, (ii) transforming the T-cells with a heterologous polynucleotide of the invention or by a vector of the invention so that they express a tumour-specific T-cell receptor (TCR) or a tumour-specific binding fragment of a TCR according to the invention; and (iii) reintroducing said transduced T-cells into the patient. There is also provided a process comprising reintroducing transduced T-cells into a patient wherein said transduced T-cells are obtained from said patient and transduced ex vivo with a heterologous polynucleotide of the invention or by a vector of the invention so that they express a tumour-specific T-cell receptor (TCR) or a tumour-specific binding fragment of a TCR according to the invention. The patient in question suitably is a cancer patient particularly a human cancer patient.

The T-cells may optionally be expanded before or (more preferably) after step (ii) above. The T-cells may be expanded by multiple methods, e.g. by treatment with IL-2 and/or antibodies against CD3 and CD28.

In a further embodiment which is a variant of the aforementioned processes, the transduced T-cells which are administered to the patients were not originally from the same patient (i.e. they are allogeneic cells).

The T-cells that are introduced into the patient after transformation may be polyclonal or monoclonal. In the latter case, a particular transduced clone is selected for expansion before administering the cells to the patient.

There is also provided a method of treatment of cancer comprising administering to a patient in need thereof transduced T-cells wherein the transduced T-cells are T-cells which have been obtained from said patient and transduced ex vivo with a heterologous polynucleotide of the invention or by a vector of the invention so that they express a tumour-specific T-cell receptor (TCR) or a tumour-specific binding fragment of a TCR according to the invention.

There are also provided transduced T-cells for use in the treatment of cancer wherein the transduced T-cells are T-cells which have been obtained from said patient and transduced ex vivo with a heterologous polynucleotide of the invention or by a vector of the invention so that they express a tumour-specific T-cell receptor (TCR) or a tumour-specific binding fragment of a TCR according to the invention.

There is also provided use of the aforementioned transduced T-cells in the manufacture of a medicament for the treatment of cancer.

As noted above, since a complete TCR comprises a γ chain and a δ chain, the T-cells may (i) be transduced with two polynucleotides each encoding a chain of the TCR or with two vectors each comprising a polynucleotide encoding a chain of the TCR or (ii) may be transduced with a polynucleotide (e.g. a transposon) encoding both chains of the TCR or with a vector comprising a polynucleotide encoding both chains of the TCR. Yet further, the two chains of the TCR may be linked by a cleavable peptide linker (e.g. a linker that cleaves in a T-cell) and so the T-cells may be transduced with a polynucleotide which encodes both chains of the TCR and the linker or with a vector comprising a polynucleotide which encodes both chains of the TCR and the linker such that the two chains of the TCR are produced in the T-cell.

In an embodiment, expression of the endogenous TCR in the T-cell is inhibited or prevented. Various methods can be employed to silence the genes encoding the γ and δ chains of the endogenous TCR. For example, the genes encoding the endogenous TCR may be silenced at the RNA level by an siRNA approach. Thus, an inhibitory or interfering RNA, such as a short hairpin RNA or other double stranded RNA comprising a region complementary to the gene to be silenced (e.g. comprising a sequence of 18-25 preferably 20-25 based paired nucleotides having complementarity to a region in the gene to be silenced) may be administered to the T-cells. The same vector to that of the TCR or a different one may be used. Alternatively, the genes encoding the endogenous TCR may be deleted or disrupted e.g. using a CRISPR approach. Thus, the elements of CRISPR/Cas9 including the Cas9 enzyme and an appropriate guide RNA selected for disruption or deletion of the genes in question may be administered to the T-cells, for example electroporation of a complex containing the Cas9 enzyme and the guide RNA. Alternatively, the Cas9 enzyme and the guide RNA components could be transduced with a vector.

### Fusion Proteins

As discussed above, it may be convenient to produce the TCR as a fusion protein comprising the γ and δ chains (or fragments thereof comprising at least one CDR, preferably 3 CDRs per chain) which is cleaved to form the respective γ and δ chains in a cell (e.g. a T cell). Hence the two chains or fragments thereof are connected by a cleavable linker.

Suitable cleavable linkers include self-cleaving linkers of the 2A family, which can include P2A, E2A, F2A and T2A. P2A is derived from porcine teschovirus-1 2A, E2A is derived from equine rhinitis A virus, F2A is derived from foot-and-mouth disease virus, T2A is derived from Thosea asigna virus 2A. A linking peptide such as GSGSG (SEQ ID NO: 36) may be used N-terminal to the 2A self-cleaving linker. See Example 1(i) for an example embodiment.

The sequence of an exemplary fusion protein comprising a TCR γ chain (SEQ ID NO: 15) and a TCR δ chain (SEQ ID NO: 16 connected via a GSGSG linking peptide and self-cleaving T2A linker (indicated by underlining) is given below:

(SEQ ID NO: 24)
MRWALAVLLAFLSPASQKSSNLEGRTKSVTRQTGSSAEITCDLTVTNTFYI
HWYLHQEGKAPQRLLYYDVSTARDVLESGLSPGKYYTHTPRRWSWILRLQN
LIENDSGVYYCATWDRRDYKKLFGSGTTLVVTDKQLDADVSPKPTIFLPSI
AETKLQKAGTYLCLLEKFFPDVIKIHWQEKKSNTILGSQEGNTMKTNDTYM
KFSWLTVPEKSLDKEHRCIVRHENNKNGVDQEIIFPPIKTDVITMDPKDNC
SKDANDTLLLQLTNTSAYYMYLLLLLKSVVYFAIITCCLLRRTAFCCNGEK
SGSGSGEGRGSLLTCGDVEENPGPMLFSSLLCVFVAFSYSGSSVAQKVTQA
QSSVSMPVRKAVTLNCLYETSWWSYYIFWYKQLPSKEMIFLIRQGSDEQNA
KSGRYSVNFKKAAKSVALTISALQLEDSAKYFCALGVLPTVTGGGLIFGKG
TRVTVEPRSQPHTKPSVFVMKNGTNVACLVKEFYPKDIRINLVSSKKITEF
DPAIVISPSGKYNAVKLGKYEDSNSVTCSVQHDNKTVHSTDFEVKTDSTDH
VKPKETENTKQPSKSCHKPKAIVHTEKVNMMSLTVLGLRMLFAKTVAVNFL
LTAKLFF

Thus, an exemplary sequence of a T2A self-cleaving linker is EGRGSLLTCGDVEENPGP (SEQ ID NO: 37).

The sequence of an exemplary polynucleotide that encodes a fusion protein comprising a TCR γ chain (SEQ ID NO: 15) and a TCR δ chain (SEQ ID NO: 16 connected via a self-cleaving 2A linker (indicated by underlining) and which has been codon optimised is given below:

(SEQ ID NO: 23)
ATGAGGTGGGCCCTGGCCGTGCTCCTGGCTTTTCTCAGCCCTGCCTCCCAG
AAGAGCTCCAACCTGGAGGGAAGGACCAAGTCCGTGACCAGACAGACAGGC
AGCAGCGCCGAGATCACCTGTGATCTGACCGTGACCAATACCTTTTACATC
CATTGGTACCTGCATCAGGAGGGCAAGGCCCCTCAAAGGCTGCTCTACTAC
GATGTCTCCACCGCCAGAGATGTGCTGGAATCCGGACTGAGCCCCGGCAAA
TATTACACCCATACCCCCAGGAGGTGGTCCTGGATCCTGAGACTGCAGAAT
CTGATCGAGAATGACAGCGGCGTGTACTACTGCGCCACCTGGGACAGAAGG
GATTACAAGAAGCTGTTCGGCAGCGGCACAACCCTGGTCGTGACCGACAAG
CAGCTGGACGCCGATGTGAGCCCTAAGCCCACAATCTTTCTGCCCTCCATC
GCTGAGACCAAACTGCAGAAGGCCGGCACATATCTCTGTCTCCTGGAGAAG
TTTTTTCCCGACGTGATCAAGATCCATTGGCAGGAGAAGAAGAGCAACACC
ATTCTCGGCAGCCAGGAAGGCAATACCATGAAAACAAATGACACATACATG
AAGTTTTCCTGGCTGACAGTCCCCGAGAAAAGCCTCGACAAGGAACACAGG
TGCATCGTCAGGCACGAGAACAATAAGAACGGCGTGGACCAAGAGATCATT
TTTCCTCCTATCAAAACAGACGTCATCACAATGGATCCCAAGGACAACTGC
AGCAAGGATGCCAATGACACCCTGCTCCTGCAGCTGACCAACACCAGCGCC
TACTACATGTACCTGCTGCTGCTCCTGAAGTCCGTGGTGTACTTCGCTATC
ATCACATGCTGCCTGCTGAGAAGAACCGCCTTCTGTTGCAACGGAGAGAAG
TCCGGATCCGGATCCGGCGAGGGACGCGGAAGCCTGCTGACCTGTGGCGAC
GTGGAGGAAAACCCCGGGCCCATGCTGTTCTCCAGCCTGCTGTGTGTGTTT
GTCGCCTTCAGCTATAGCGGCTCCAGCGTGGCCCAGAAGGTGACCCAGGCC
CAGTCCAGCGTGAGCATGCCCGTGAGGAAGGCCGTCACCCTGAACTGTCTG
TACGAGACCAGCTGGTGGAGCTACTACATCTTCTGGTACAAGCAGCTGCCC
AGCAAGGAGATGATCTTTCTGATCAGGCAGGGCTCCGATGAACAGAATGCC
AAGAGCGGCAGGTACTCCGTGAACTTCAAGAAGGCCGCCAAAAGCGTCGCC
CTGACCATCTCCGCCCTGCAGTTAGAAGACTCCGCCAAGTACTTTTGTGCC
CTGGGAGTGCTGCCTACCGTGACCGGAGGCGGCCTGATCTTTGGCAAAGGA
ACCAGGGTCACCGTGGAGCCTAGATCCCAACCCCACACCAAACCCAGCGTG
TTCGTGATGAAAAACGGCACCAACGTGGCCTGTCTCGTCAAGGAATTTTAC
CCCAAAGACATCAGGATCAACCTCGTGTCCTCCAAGAAGATCACCGAGTTC
GACCCTGCCATCGTGATCTCCCCTAGCGGAAAGTACAACGCCGTGAAACTG
GGAAAGTACGAGGATTCCAACTCCGTGACCTGCTCCGTCCAACATGACAAC
AAGACCGTGCACAGCACCGACTTCGAGGTGAAGACAGATAGCACCGATCAC
GTGAAGCCCAAGGAAACAGAGAACACCAAGCAGCCTAGCAAGTCCTGCCAC
AAGCCCAAGGCCATCGTCCACACCGAGAAAGTCAACATGATGAGCCTGACC
GTGCTGGGCCTGAGGATGCTGTTTGCCAAGACCGTCGCCGTGAATTTCCTG
CTGACCGCCAAACTCTTCTTCCT

The sequence of SEQ ID NO: 23 does not include a stop codon. A suitable stop codon e.g. TAA will typically be included at the C terminus in a construct including this sequence.

### Multimers

The TCR of the invention, particularly in its soluble form, may be employed in multimeric form, for example a tetramer, pentamer. The multimer may also be a dextramer. Such multimers may be used for identifying and/or capturing cells that express the SCNN1A gene product target on the surface. For example, the TCR of the invention may be provided with a biotin tag and complexed in multimeric form to streptavidin. Streptavidin may be complexed to a detection signal e.g. a fluorophore.

### Bispecifics

According to a yet further aspect of the invention there is provided a pharmaceutical composition or bispecific comprising said TCR or cell or clone or vector.

Alternatively still, said TCR (or tumour-specific binding fragments thereof) may form part of a bispecific wherein said bispecific includes said TCR, for the purpose of binding to its ligand on a cancer cell, and also an immune cell activating component or ligand that binds and so activates an immune cell such as a Killer T-cell.

In an embodiment, there is provided a bispecific construct comprising the TCR or tumour-specific binding fragment of the invention (such as a soluble TCR) and an immune cell activating component or ligand (such as an antibody protein) that binds to and activates an immune cell such as T-cell e.g. a CD8+ T-cell. For example, the immune cell activating component or ligand activates an immune cell via binding to CD3.

For example, one particular bispecific construct comprises the polypeptide sequences of a soluble TCR of the invention and an antibody protein (particularly an agonist antibody protein) that binds to CD3.

Said antibody protein is suitably an antigen-binding domain of an antibody such as a VH (variable heavy domain from a 4 chain antibody) or a VHH (variable heavy domain from a 2 chain (heavy chain only) antibody such as from a Camelid) or is a scFv (i.e. a fusion protein comprising the variable regions of the light and heavy chains of an antibody connected by a short (e.g. 10-25 amino acids) linker).

A number of anti-CD3 agonist antibodies are available in the prior art. For example, blinatumomab which is an approved bispecific T cell engager (BiTE) product consists of a CD19-targeting antibody (heavy chain scFv) connected to a CD3-targeting agonist antibody (light chain scFv). The CD3-targeting agonist antibody (light chain scFv) component of blinatumomab could therefore be used. The other BiTE products solitomab, pasotuxizumab and ertumaxomab also comprise a CD3-targeting agonist antibody component which could be used in a bispecific of the present invention. The TCR-based bispecific tebentafusp also comprises a CD3-targeting agonist antibody component which could be used in a bispecific of the present invention.

A linker may be provided to link a chain of the TCR to the other part of the bispecific construct e.g. a non-immunogenic linker sequence such as GGGGS (SEQ ID NO: 25).

In an embodiment, the bispecific construct comprises an antibody protein which binds to a SCNNA1 gene product expressed on a tumour cell. For example, it comprises an antibody protein (e.g. an scFv derived from a monoclonal antibody) which binds to a SCNNA1 gene product expressed on a tumour cell and an antibody protein (particularly an agonist antibody protein) that binds to CD3.

More generally, there is provided a fusion protein comprising the TCR or tumour-specific binding fragment of the invention and a heterologous protein providing the immune cell stimulating and/or activating activity.

There is also provided a polynucleotide encoding said bispecific, bispecific construct or fusion protein. Said polynucleotide is suitably a DNA, including single- or double-stranded DNA and straight-chain or circular DNA.

### Compositions

The invention provides a pharmaceutical composition comprising the TCR or tumour-specific binding fragment thereof, polynucleotide, vector, T-cell, T-cell clone, bispecific, bispecific construct or fusion protein of the invention and a pharmaceutically acceptable carrier. Suitably the pharmaceutical composition is formulated under sterile conditions and is suitable for parenteral administration. For parenteral administration, the carrier preferably comprises water and may contain buffers for pH control, stabilising agents e.g., surfactants and amino acids and tonicity modifying agents e.g., salts and sugars.

### Cancer Treatment

In a preferred embodiment said pharmaceutical composition or bispecific is used to treat any cancer, ideally colorectal, lung, kidney, prostate, bladder, cervical, melanoma (e.g. skin melanoma), bone, breast, blood cancer (e.g. leukemia), brain, pancreas, testicle, ovary, head/neck, liver, bladder, thyroid, and uterine cancer, particularly melanoma (skin), kidney, colorectal, breast, blood (leukemia), lung, cervical and bone cancer.

According to a yet further aspect of the invention there is provided the use of said TCR (or tumour-specific binding fragment thereof) or cell or clone or polynucleotide or vector to treat cancer.

According to a yet further aspect of the invention there is provided a method of treating cancer comprising administering said TCR or cell or clone or vector to an individual to be treated.

More generally, there is provided a method of treating cancer in a subject comprising administering a therapeutically effective amount of the T-cell, T-cell clone, bispecific, bispecific construct, fusion protein or pharmaceutical composition of the invention to the subject.

Except where the context indicates otherwise, reference herein to “tumour” includes a reference to “cancer”.

Said cancer may include solid tumours and blood cancers but in particular colorectal, lung, kidney, prostate, bladder, cervical, melanoma (e.g. skin melanoma), bone, breast, blood cancer (e.g. leukaemia), brain, pancreas, testicle, ovary, head/neck, liver, bladder, thyroid, and uterine cancer, particularly melanoma (skin), kidney, colorectal, breast, blood (leukemia), lung, cervical and bone cancer.

According to a yet further aspect of the invention there is provided the use of said TCR or cell or clone or vector in the manufacture of a medicament to treat cancer.

More generally, there is provided use of a T-cell, T-cell clone, bispecific, bispecific construct, fusion protein or pharmaceutical composition in the manufacture of a medicament to treat cancer.

There is also provided a T-cell, T-cell clone, bispecific, bispecific construct, fusion protein or pharmaceutical composition for use in the treatment of cancer.

Equally well, it is also envisaged that all embodiments of the present invention that may be used to treat cancer may potentially be used to prevent cancer. Thus, corresponding methods and uses and substances for use to prevent cancer are provided as an aspect of the invention.

### Combinations

According to a yet further aspect of the invention there is provided a combination therapeutic for the treatment of cancer comprising:
a) said TCR or cell or clone or vector in combination with
b) a further cancer therapeutic agent.

More generally, there is provided a pharmaceutical composition comprising:
a) the T-cell, T-cell clone, pharmaceutical composition, bispecific, bispecific construct or fusion protein of the invention; and
b) an anti-tumour agent.

Alternatively, the T-cell, T-cell clone, pharmaceutical composition, bispecific, bispecific construct or fusion protein of the invention may be administered separately, simultaneously or sequentially with an anti-tumour agent.

Further cancer therapeutic agents/anti-tumour agents that may be included in a combination therapy include immune check point inhibitors e.g. selected from PD-1 inhibitors, such as pembrolizumab, (Keytruda) and nivolumab (Opdivo), PD-L1 inhibitors, such as atezolizumab (Tecentriq), avelumab (Bavencio) and durvalumab (Imfinzi) and CTLA-4 inhibitors such as ipilimumab (Yervoy), other immune stimulants such as interferons (e.g. interferon α, β or γ), steroids e.g. prednisolone and alkylating agents such as platinum-based anti-neoplastic agents e.g. cisplatin, carboplatin and oxaliplatin.

In a preferred method of the invention said TCR, cell, clone or vector is administered in combination with an anti-tumour agent such as, but not limited to, a bispecific.

### Targeting Cancer Cells Expressing the SCNN1a Gene Product

More generally, the invention provides a TCR or a tumour-specific binding fragment of a TCR or bispecific construct that binds to a SCNNA1 gene product expressed on a tumour cell.

The invention also provides bispecific construct comprising an antibody protein which binds to a SCNNA1 gene product expressed on a tumour cell.

Said antibody protein is suitably an antigen-binding domain of an antibody such as a VH (variable heavy domain from a 4 chain antibody) or a VHH (variable heavy domain from a 2 chain (heavy chain only) antibody such as from Camelid) or is a scFv (i.e. a fusion protein comprising the variable regions of the light and heavy chains of an antibody connected by a short (e.g. 10-25 amino acids) linker).

The SCNNA1 gene product is, for example, selected from the SCNNA1 gene product isoforms having sequences of SEQ ID NOs. 29-34.

The TCR or a tumour-specific binding fragment of a TCR or bispecific construct may in particular bind to the extracellular domain of said isoform.

There is also provided a polynucleotide encoding the TCR or a tumour specific binding fragment of a TCR or bispecific construct as aforementioned. The polynucleotide may for example be a chimeric polynucleotide comprising a gene encoding the TCR or tumour specific binding fragment of a TCR or bispecific construct and a heterologous promoter or other transcription control element operably linked thereto.

There is also provided a vector for delivery of a polynucleotide to cells comprising a polynucleotide as aforementioned. The vector may for example be a viral vector e.g. a lentiviral vector.

There is also provided a T-cell expressing the TCR or tumour-specific binding fragment as aforementioned.

There is also provided a pharmaceutical composition comprising the TCR or tumour-specific binding fragment of a TCR, bispecific construct, polynucleotide or vector as aforementioned and a pharmaceutically acceptable carrier. The pharmaceutical composition may for example be formulated under sterile conditions and be suitable for parenteral administration.

There is also provided a method of treating cancer in a subject comprising administering a therapeutically effective amount of the TCR or tumour-specific binding fragment of a TCR, bispecific construct, polynucleotide, vector, T cell or pharmaceutical composition as aforementioned to the subject. Said cancer may for example be selected from the group consisting colorectal cancer, lung, kidney, prostate, bladder, cervical, skin melanoma, bone, breast, blood cancer, brain, pancreas, testicle, ovary, head/neck, liver, bladder, thyroid and uterine.

In the claims which follow and in the preceding description of the invention, except where the context requires otherwise due to express language or necessary implication, the word “comprises”, or variations such as “comprises” or “comprising” is used in an inclusive sense i.e. to specify the presence of the stated features but not to preclude the presence or addition of further features in various embodiments of the invention.

All references, including any patent or patent application, cited in this specification are hereby incorporated by reference. No admission is made that any reference constitutes prior art. Further, no admission is made that any of the prior art constitutes part of the common general knowledge in the art.

Preferred features of each aspect of the invention may be as described in connection with any of the other aspects.

Other features of the present invention will become apparent from the following examples. Generally speaking, the invention extends to any novel one, or any novel combination, of the features disclosed in this specification (including the accompanying claims and drawings). Thus, features, integers, characteristics, compounds or chemical moieties described in conjunction with a particular aspect, embodiment or example of the invention are to be understood to be applicable to any other aspect, embodiment or example described herein, unless incompatible therewith.

Moreover, unless stated otherwise, any feature disclosed herein may be replaced by an alternative feature serving the same or a similar purpose.

Throughout the description and claims of this specification, the singular encompasses the plural unless the context otherwise requires. In particular, where the indefinite article is used, the specification is to be understood as contemplating plurality as well as singularity, unless the context requires otherwise.

Further clauses which define the invention are set out below:
1. A tumour-specific T-cell receptor (TCR), or a fragment thereof, characterised by at least one complementarity-determining region comprising or consisting of (CDR) CATWDRRDYKKLF (SEQ ID NO: 1) and/or CALGVLPTVTGGGLIF (SEQ ID No: 2) or a CDR that shares at least 88% identity with either CDR (i.e. SEQ ID NO: 1 or 2).
2. The tumour-specific T-cell receptor (TCR) according to claim 1 wherein said wherein said TCR comprises both of the said CDRs.
3. The tumour-specific T-cell receptor (TCR) according to claim 1 or claim 2 wherein said TCR comprises or consists of one or more, including any combination, of the following complementarity-determining regions:

SEQ ID NO: 3
VTNTFY (CDR1g)
SEQ ID NO: 4
YDVSTARD (CDR2g)
SEQ ID NO: 5
TSVWVSYY (CDR1d)
or
SEQ ID NO: 6
QGS (CDR2d).

4. The tumour-specific T-cell receptor (TCR) according to any one of claims 1-3 wherein said TCR is a gamma chain comprising or consisting of:

(SEQ ID NO: 7)
SSNLEGRTKSVTRQTGSSAEITCDLTVTNTFYIHWYLHQEGKAPQRLLYYD
VSTARDVLESGLSPGKYYTHTPRRWSWILRLQNLIENDSGVYYCATWDRRD
YKKLFGSGTTLVVTDKQLDADVSPKPTIFLPSIAETKLQKAGTYLCLLEKF
FPDVIKIHWQEKKSNTILGSQEGNTMKTNDTYMKFSWLTVPEKSLDKEHRC
IVRHENNKNGVDQEIIFPPIKT

or a gamma chain that shares at least 88% identity therewith.
5. The tumour-specific T-cell receptor (TCR) according to any one of claims 1-3 wherein said TCR is a delta chain comprising or consisting of:

(SEQ ID NO: 8)
AQKVTQAQSSVSMPVRKAVTLNCLYETSWWSYYIFWYKQLPSKEMIFLIRQ
GSDEQNAKSGRYSVNFKKAAKSVALTISALQLEDSAKYFCALGVLPTVTGG
GLIFGKGTRVTVEPNSQPHTKPSVFVMKNGTNVACLVKEFYPKDIRINLVS
SKKITEFDPAIVISPSGKYNAVKLGKYEDSNSVTCSVQHDNKTVHSTDFEV
KTDST

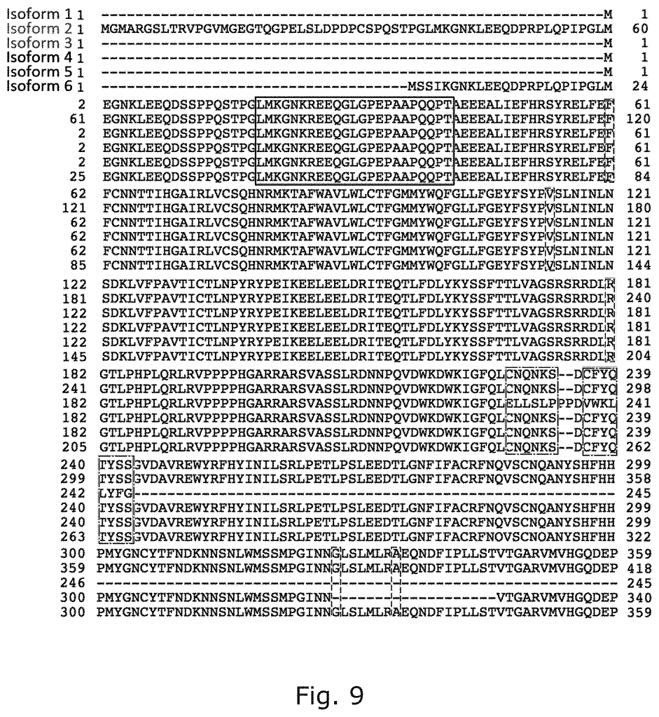

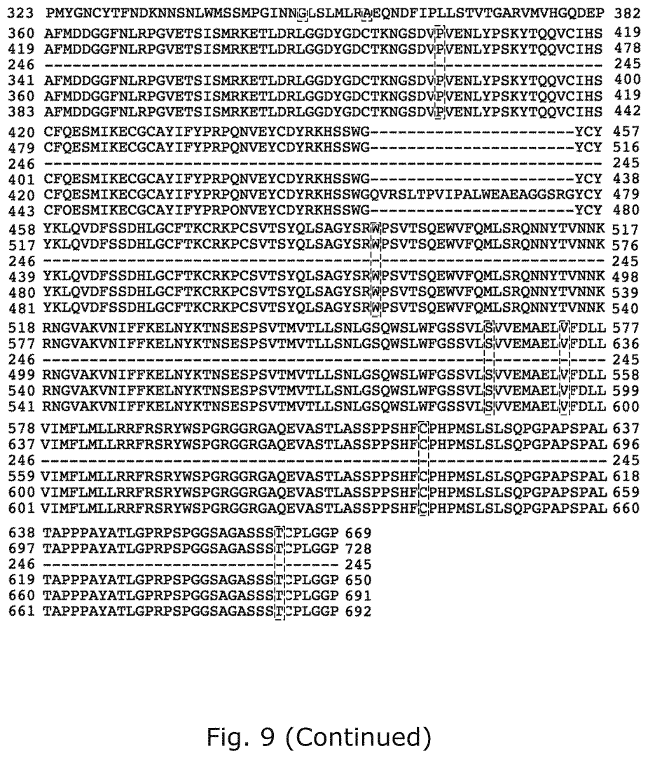

or a delta chain that shares at least 88% identity therewith.
6. The tumour-specific T-cell receptor (TCR) according to any one of claims 1-5 wherein said CDR of said TCR additionally or alternatively comprises or consists of a gamma chain sequence that is CALWEVDYKKLF (SEQ ID NO: 9); and/or a delta chain sequence that is CALGEPVLFAVRGLIF (SEQ ID NO: 10) and/or CACDLLGDRYTDKLIF (SEQ ID NO: 11); or a CDR that shares at least 88% identity with said gamma chain sequence or said delta chain sequence.
7. The tumour-specific T-cell receptor (TCR) according to any one of claims 1-6 wherein said TCR is soluble.
8. A T-cell expressing said TCR according to any one of claims 1-7.
9. A T-cell clone expressing said TCR according to any one of claims 1-7.
10. The T-cell clone according to claim 9 wherein said clone is a SW.3G1 clone.
11. A vector encoding the TCR according to anyone of claims 1-7.
12. A pharmaceutical composition or immunogenic agent or bispecific or vaccine comprising said TCR according to any one of claims 1-7 or said cell according to claim 8 or said clone according to claim 9 or 10 or said vector according to claim 11.
13. The TCR according to any one of claims 1-7 or the said cell according to claim 8 or the said clone according to claim 9 or 10 or the said vector according to claim 11 or the said pharmaceutical composition or immunogenic agent or bispecific or vaccine according to claim 12 for use in the treatment of cancer.
14. The TCR, the cell, the clone, the vector, the pharmaceutical composition or immunogenic agent or bispecific or vaccine according to claim 13 wherein said cancer is selected from the group comprising or consisting of colorectal cancer, lung, kidney, prostate, bladder, cervical, melanoma (skin), bone, breast, blood cancer, brain, pancreas, testicle, ovary, head/neck, liver, bladder, thyroid, and uterine.
15. A method of treating cancer comprising administering the said TCR according to any one of claims 1-7 or the said cell according to claim 8 or the said clone according to claim 9 or 10 or the said vector according to claim 11 or the said pharmaceutical composition or immunogenic agent or bispecific or vaccine according to claim 12 to an individual to be treated.
16. The method according to claim 15 wherein said cancer is selected from the group comprising or consisting of colorectal cancer, lung, kidney, prostate, bladder, cervical, melanoma (skin), bone, breast, blood cancer, brain, pancreas, testicle, ovary, head/neck, liver, bladder, thyroid, and uterine.
17. The method according to claim 15 or 16 wherein said TCR, cell, clone, vector, pharmaceutical composition, immunogenic agent, bispecific or vaccine is administered in combination with an anti-tumour agent.
18. Use of said TCR according to claims 1-7 or the said cell according to claim 8 or the said clone according to claim 9 or 10 or the said vector according to claim 11 in the manufacture of a medicament to treat cancer.
19. A combination therapeutic for the treatment of cancer comprising:
   a) the said TCR according to claims 1-7 or the said cell according to claim 8 or the said clone according to claim 9 or 10 or the said vector according to claim 11 or the said pharmaceutical composition or immunogenic agent or bispecific or vaccine according to claim 12 in combination with;
   b) a further cancer therapeutic agent.
20. A TCR or polypeptide or bispecific or antibody, or a fragment of said antibody, that binds to at least one of the SCNNA1 gene product isoforms shown in FIG. 9.
21. The TCR or polypeptide or bispecific or antibody, or a fragment of said antibody, according to claim 20 that binds to the extracellular domain of said isoform.
22. The TCR or polypeptide or bispecific or antibody, or a fragment of said antibody, according to claim 20 or claim 21 wherein said antibody is monoclonal.
23. At least one SCNNA1 gene product isoform shown in FIG. 9, or a fragment thereof, that binds to a TCR according to any one of claims 1-7.
24. A TCR, cell, clone, vector, pharmaceutical composition, immunogenic agent, bispecific or vaccine or polypeptide or antibody, or a fragment of said antibody, as substantially here in described.

Embodiments of the present invention will now be described by way of example only with reference to the following wherein:

FIGS. 1A-B show how a γδ T-cell line reactive to autologous and non-autologous lymphoblastoid cell lines (LCLs) was clonotyped and found to express a TCR comprised of the TRGV3 and TRDV1 genes with the CDR3s CATWDRRDYKKLF (SEQ ID NO: 1) and CALGVLPTVTGGGLIF (SEQ ID NO: 2), respectively. A clone was grown by limited dilution that expressed this TCR and named SW.3G1. (FIG. 1A) Purified γδ T-cells from a healthy donor, 9909, were primed (day 0) and re-stimulated (day 14) with a pool of LCLs from three donors (0439, pt146 and Hom-2). On day 28 the T-cell line was incubated with the LCLs used for priming and also autologous LCL-9909 for 4 h with activation assessed by inclusion of TAPI-0, anti-CD107a and anti-TNFα antibodies. The activated cells were sorted by flow cytometry and the T-cell receptors (TCRs) analysed by next generation sequencing. Pie charts depict the proportion of the displayed TCR chains and CDR3s (complementarity determining regions) that were present in the sorted cells. The percentage of activated cells for the flow cytometry plots is shown above each gate. T-cell clone SW.3G1 obtained from the lines expresses the highlighted TCR chains, with the TCR extracellular domain less the connecting peptide sequences shown in FIG. 2 (SEQ ID NOs: 7 and 8; δ chain mutant version). (FIG. 1B) Clone SW.3G1 was phenotyped with the antibodies and confirmed to express a TCRδ1 chain. SW.3G1 did not express an αβTCR or CD8 or CD4 glycoproteins associated with recognition of conventional peptide-HLA antigens.

FIG. 2 shows the T-cell receptor sequence of the γ and δ TCR chains of clone SW.3G1 (extracellular regions less the connecting peptide only, mutant version of δ chain, Example 2a, SEQ ID NOs: 7 and 8). The mRNA structures (top) show that for each chain CDR1 and CDR2 are encoded in the germline. CDR3 is the product of junctional diversity at V-J joins of T cell receptor (TCR)-γ chain and V-D-J joins in TCR-δ chain. CDR3 is consequently hypervariable. The order adopted for the CDR loops is maintained throughout the figure. The panel on the right shows the expected protein fold. TCRs adopt similar tertiary structures that position the complementarity-determining regions (CDR) loops at the membrane distal end of the molecules. Together the six CDR loops form the antigen binding site.

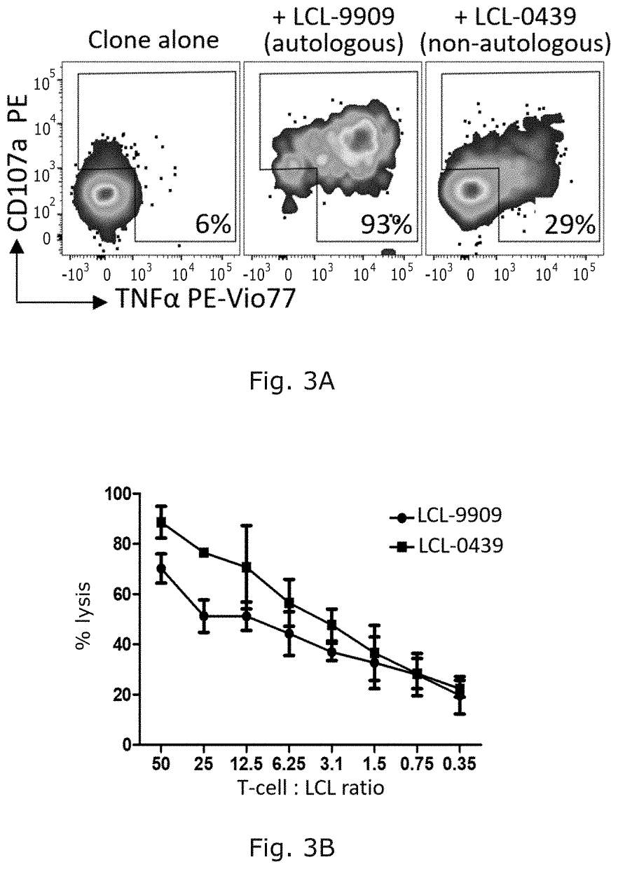

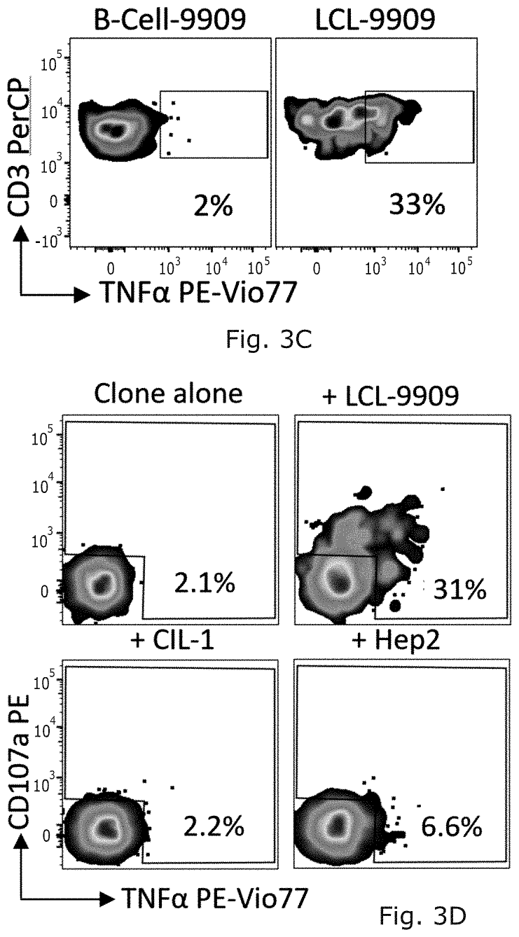

FIGS. 3A-D show SW.3G1 can recognize and kill autologous and non-autologous LCLs, but not healthy cells of various tissue origins. (FIG. 3A) Co-incubation of SW.3G1 with LCLs for 4 h, with activation assessed by inclusion of TAPI-0, anti-CD107a and anti-TNFα antibodies. (FIG. 3B) 6.5 h chromium release cytotoxicity assay using the same LCLs as in (FIG. 3A). (FIG. 3C) Autologous healthy B-cells magnetically purified directly ex-vivo from donor 9909 were used in activations assays as in FIG. 3A, with LCL-9909 used as a positive control for activation. (FIG. 3D) Activation assays as in FIG. 3A, using LCL-9909 and the healthy cell lines, CIL-1 (non-pigmented ciliary epithelium) and Hep2 (hepatocyte). Percentage of gated cells is shown.

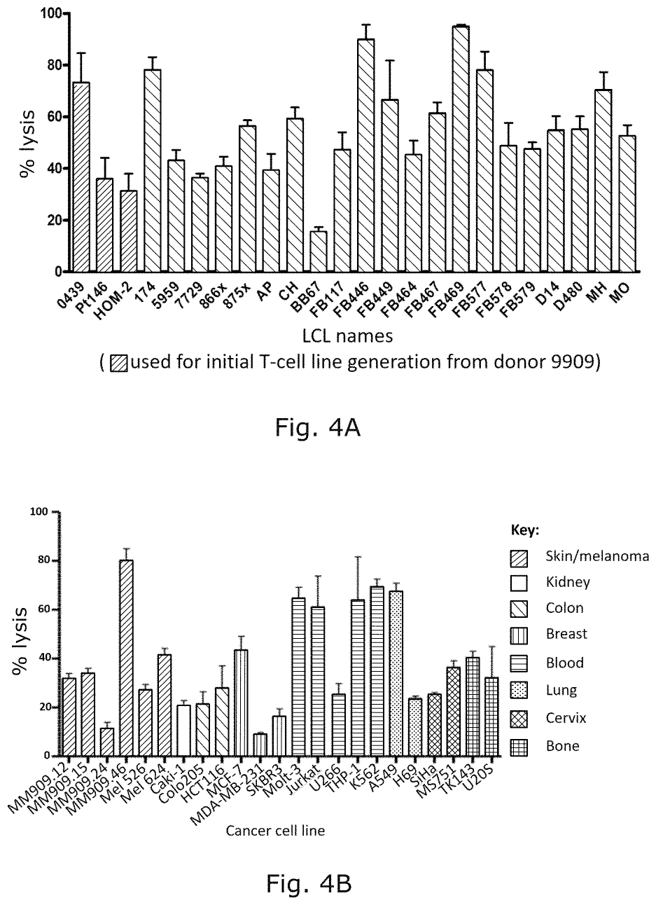

FIGS. 4A-B show SW.3G1 mediated lysis of LCLs from multiple donors that share no common HLA and an array of cancer cell lines from different tissues. SW.3G1 was used in 6.5 h chromium release cytotoxicity assays. (FIG. 4A) A panel of lymphoblastic cell lines (LCLs) from 24 donors (named on the x-axis). The first three bars (narrow hatch) depict LCLs that were used to generate the T-cell lines from donor 9909 from which SW.3G1 was cloned. T-cell to LCL ratio of 1:1. (FIG. 4B) SW.3G1-mediated killing of panel of cancer cell lines (named on the x-axis) of different tissue origin (key) at a T-cell to cancer cell ratio of 10:1. Note that the key for (FIG. 4B) does not correspond to (FIG. 4A).

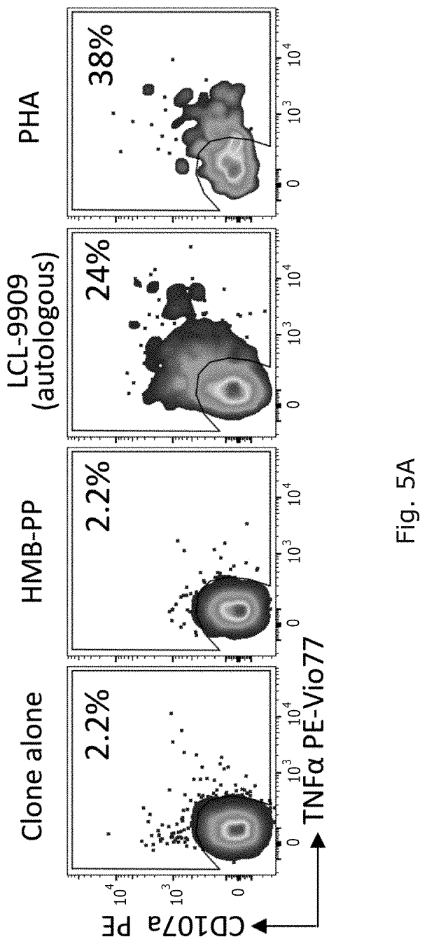

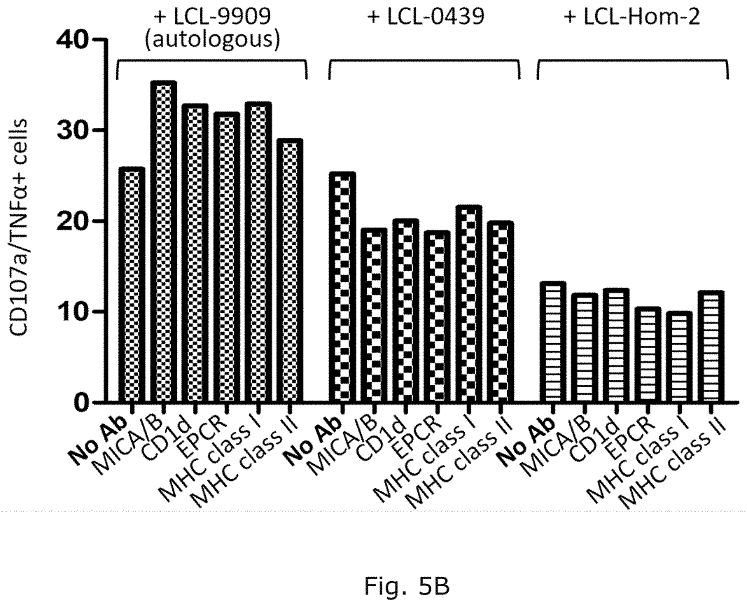

FIGS. 5A-B show that SW.3G1 does not recognize target cells by known mechanisms. (FIG. 5A) γδ SW.3G1 clone was co-incubated for 4 h with HMB-PP, the lymphoblastic cell line (LCL)-9909 and phytohaemagluttinin (PHA). T-cells were also incubated alone. T-cell activation was assessed by inclusion of TAPI-0, anti-CD107a and anti-TNFα antibodies, with the percentage of activated cells shown above the gated cells. (FIG. 5B) Using the same activation assay as in (FIG. 5A), SW.3G1 was incubated with LCLs that had been pre-labelled with antibodies (Abs) that bind the proteins named on the x-axis. SW.3G1 was also incubated with the LCLs without Ab (no Ab control). The percentage of reactivity is shown graphically (y-axis). MICA/B (Major Histocompatibility Complex (MHC) Class-I related chain A/B) and EPCR (Endothelial protein C receptor). Anti-MHC class I and II Abs were also included.

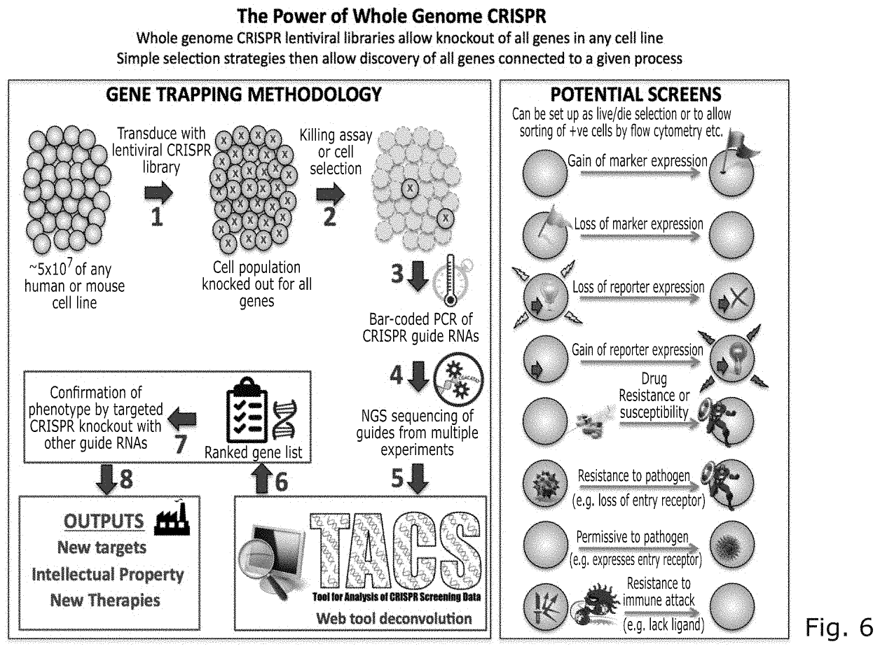

FIG. 6 shows the whole genome CRISPR/Cas9 approach used to identify candidate genes/proteins involved in target cell recognition by SW.3G1.

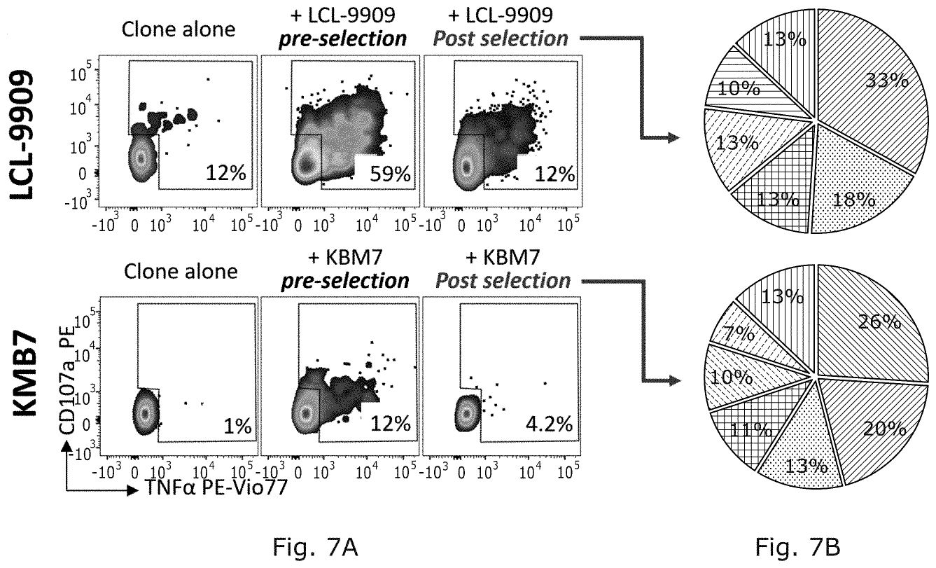

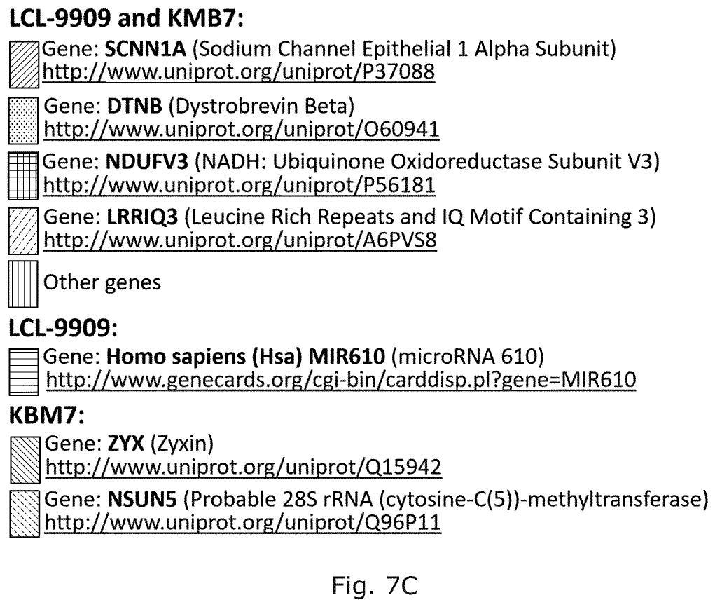

FIGS. 7A-C show the results of the whole genome CRISPR/Cas9 approach that identified multiple candidate genes for target cell recognition by SW.3G1. (FIG. 7A) Autologous LCL-9909 and cancer cell line KBM7s were transduced with a whole genome CRISPR/Cas9 library. The libraries were put though several selections using the SW.3G1 T-cell clone to generate a target cell line that was resistant to lysis. The surviving target cells (post-selection) were tested alongside the pre-selected cell lines in activation assays with SW.3G1. Activation assessed by inclusion of TAPI-0, anti-CD107a and anti-TNFα antibodies. (FIG. 7B) Sequencing of the post-selection libraries revealed enriched guide RNAs corresponding to the key shown in (FIG. 7C), identifying genes of interest. (FIG. 7C) Candidate genes seen in both the LCL-9909 and KBM7 libraries, or seen only for LCL-9909 or KBM7s. Gene and (protein) names are shown with website links for further information. The hatched key refers to the hatching of the pie charts in (FIG. 7B).

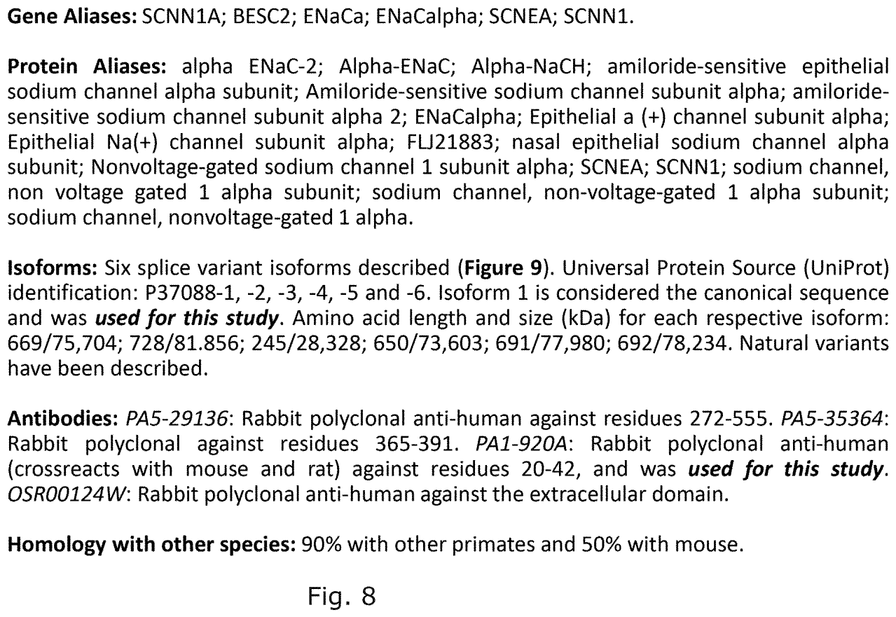

FIG. 8 shows information about the candidate gene/protein SCNN1A, which was identified by the whole genome CRISPR/cas9 library approach.

FIG. 9 shows the canonical protein sequence of SCNN1A which aligned with five expressed splice variants. Isoform 1 is the canonical sequence (Isoform 1 UniProt P37088-1). Boxed amino acids in BLACK in solid lines: region used to generate the polyclonal Ab used in this study. Boxed amino acids in dashed line: Sites of protein variants due to different amino acid residues to the ones shown. The amino acid residues of the protein variants and are not displayed here, but can be found at http://www.uniprot.org/uniprot/P37088. Boxed amino acids in alternating dash and dot lines: amino acid differences between splice isoforms.

FIGS. 10A-D show the results of experiments to validate the role of SCNN1A in target cell recognition by SW.3G1. (FIG. 10A) Schematic of the SCNN1A gene and protein, with guide RNA (gRNA) sites from the whole genome GeCKO library (gRNA-1, SEQ ID NO: 12) and a different validation gRNA SCNN1A sequence we designed (gRNA-2, SEQ ID NO: 13). Figure adapted from Chen 2014. (FIG. 10B) Long term killing assay using SW.3G1 with LCL 0.174 wild-type, gRNA-1 (GeCKO gRNA) and gRNA-2 (our gRNA) knock-out LCL.174 cells. (FIG. 10C) Western blot analysis of the breast cancer cell line MDA-MB-231 that had received the gRNA-2 (our gRNA) for SCNN1A. Wild-type cells used for comparison, with red arrow indicating the 76 kDa SCNN1A protein. (FIG. 10D) MDA-MB-231 cells from (FIG. 10C) and melanoma MM909.24, wild-type and SCNN1A knock-out (KO) cell lines used in long term killing assays with SW.3G1. Cancer cells were used as lines and selected by puromycin treatment for those expressing the gRNAs, with no subsequent cell cloning.

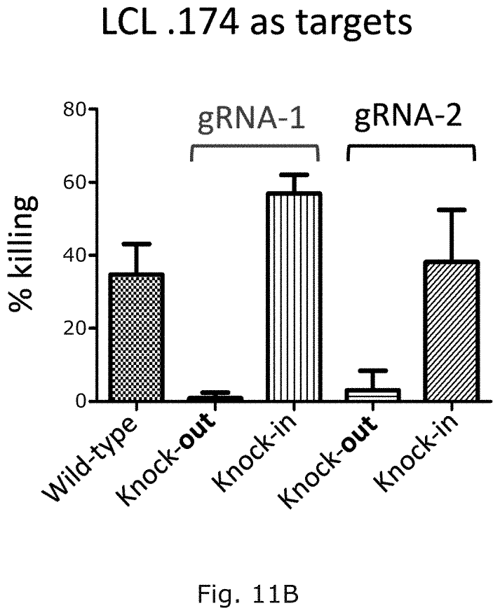

FIGS. 11A-B show that transfer of the TCR from SW.3G1 confers target cell reactivity to αβ T-cells from three healthy donors. (FIG. 11A) Purified CD8+ T-cells from three donors were co-transduced with the SW.3G1 T-cell receptor chains (marker and purification via rat(r)CD2) and a gRNA to render the recipient T-cells TCRβ chain negative (selected by puromyicn treatment) (Legut et al 2017). Purity of the cells was checked with rCD2 antibody (Ab) and anti-γδ TCR Ab. (FIG. 11B) The cells from one donor were tested in long-term killing assays (lower graph). LCL 0.174 cell lines were used: wildtype, SCNN1A knockout (using gRNA-1 and -2) and SCNN1A knock-in (KO cells that had received a codon optimized SCNN1A transgene) cells.

### EXAMPLES

### Example 1

### Methods and Materials

### (a) T-Cell Line Generation and Clonotyping

Peripheral blood mononuclear cells (PBMCs) were purified from the blood of a healthy donor (code 9909) by standard density gradient separation. The dominant population of γδ T-cells in peripheral blood express a Vγ9Vδ2 TCR and typically respond to antigens derived from bacteria. In order to enrich γδTCR+/δ2− T-cells thereby increasing the likelihood of finding cancer reactive T-cells, we modified a magnetic based purification protocol. The first adaptation was to stain the PBMCs with a PE conjugated anti-Vδ2 antibody (Ab) (clone B6, BioLegend, San Diego, Calif.). Next, γδ TCR+ T-cells were negatively enriched by positively removing γδ TCR− cells according the manufacturer's instructions (Miltheyi Biotec, Bergish Gladbach, Germany). The second adaptation involved adding anti-PE microbeads (Miltneyi Biotec) to the beads of the γδ TCR purification kit, thereby removing δ2+ cells at the same time as the γδ TCR− cells. The purified cells were co-incubated with irradiated (3000-3100 rad) LCLs from three donors that had been generated from PBMC by immortalizing B-cells with Epstein-Barr Virus (EBV). All LCLs were grown in R10 media (RPMI-1640, 10% heat-inactivated fetal calf serum, 2 mM L-glutamine, 100 U/mL Penicillin and 100 μg/mL Streptomycin, all Life Technologies, Carlsbad, Calif.) as suspension cells. After 14 days the T-cells were restimulated with irradiated LCLs from the same donors. On day 28 the T-cells were harvested and used in activation assays to assess reactivity towards LCLs. T-cells (30,000) were incubated for 4 h in 96 U well plates with an equivalent number of LCLs. 30 mM of the TNFα Processing Inhibitor-0 (TAPI-0 from Sigma Aldrich) (Haney et al., 2011), anti-CD107a Ab (H4A2, Becton Dickinson (BD), Franklin Lakes, N.J.) and anti-TNFα Ab (cA2, Miltenyi Biotec) were added to the assay media at the start of the assay, with the cells subsequently stained with the cell viability dye, Vivid (Life Technologies, 1:40 dilution in PBS then 2 μL per stain in 50 μL) and anti-CD3 antibody (Ab) (BW264/56, Miltenyi Biotec). Activated cells were sorted on a BD FACS Aria in to RLT Plus buffer (supplemented with 40 mM DTT) (Qiagen) ready for sequencing of the TCR chains. RNA was extracted using the RNEasy Micro kit (Qiagen, Hilden, Germany). cDNA was synthesized using the 5′/3′ SMARTer kit (Clontech, Paris, France) according to the manufacturer's instructions. The SMARTer approach used a Murine Moloney Leukaemia Virus (MMLV) reverse transcriptase, a 3′ oligo-dT primer and a 5′ oligonucleotide to generate cDNA templates, which were flanked by a known, universal anchor sequence. PCRs were performed using anchor-specific forward primers and reverse primers of the constant regions of the γ or δ TCR chains. The final PCR products were gel purified and prepared for next generation sequencing (Donia et al., 2017).

### (b) Clone SW.3G1 Procurement and Phenotyping

T-cells were cloned directly from the T-cell line by limiting dilution (Theaker et al., 2016). After 4 weeks of culture, 50% of each clone by culture volume was harvested and used for the activation assays with LCLs as above. Prior to performing activation assays, T-cell clones were washed and incubated for 24 h in reduced serum media. Clones that exhibited reactivity towards the LCLs were grown to sufficient numbers for TCR sequencing (below). Clone SW.3G1 was stained with Abs for surface expression of CD3 (Miltenyi Biotec), CD8 (BW135/80, Miltenyi Biotec), CD4 (M-T466, Miltenyi Biotec), αβ TCR (BW242/412, Miltenyi Biotec) and TCR Vδ1 chain (REA173, Miltenyi Biotec).

### (c) Sequencing of the SW.3G1 TCR

As above for sequencing the T-cell lines with the purified PCR products after the final PCR being cloned into Zero-Blunt TOPO and transformed into One Shot Chemically Competent E. coli cells for standard sequencing (both from Life Technologies). The sequences of the γ and δ chains of the natural SW.3G1 clone are SEQ ID NOs: 21 and 22, respectively.

### (d) SW.3G1 Recognized LCLs but not Healthy Cells

To confirm SW.3G1 reactivity towards LCLs, activation assays as above, and chromium release cytotoxicity assays were performed. Healthy B-cells were purified from donor 9909 using a PE conjugated anti-CD19 Ab (HIB19, Miltenyi Biotec) and positive capture with anti-PE microbeads (Miltenyi Biotec) and used immediately in assays. Other healthy cell lines and their proprietary culture media were obtained from Sciencell (Carlsbad, Calif.): CIL-1 (human non-pigmented ciliary epithelium) and Hep2 (human hepatocyte) were used in activation as above.

### (e) SW.3G1 Killed all Immortalized and Cancer Cell Lines Tested

LCLs and tumour cells were labelled with chromium 51 for cytotoxicity assays (Ekeruche-Makinde et al., 2012), with T-cell to target cell ratios of 1:1 (LCLs) or 10:1 (cancer cells). LCLs were maintained as above. Cancer cells lines (ATCC® reference for background and culture information)/tissue of origin: SiHa (HTB-35) and MS751 (HTB-34)/cervical; MCF7 (HTB-22), MDA-MB-231 (CRM-HTB-26) and SKBR3 (HTB-30)/breast; TK143 (CRL-8303) and U20S (HTB-96)/bone; HCT-116 (CCL-247) and Colo205 (CCL-222)/colon; Jurkat (TIB-152), K562 (CCL-243), THP-1 (TIB-202), U266 (TIB-196) and Molt-3 (CRL-1552)/blood; Caki-1 (HTB-46)/kidney; A549 (CCL-185) and H69 (HTB-119)/lung. MM909.11, MM909.12, MM909.15, MM909.46 and MM909.24 are skin melanomas obtained from cancer patients treated at the Center for Cancer Immune Therapy (CCIT, Herlev Hospital, Copenhagen, Denmark). The ‘MM’ cell lines and melanomas Mel 526 and Mel 624 were maintained as adherent cells in R10, passaged once weekly or when required, aiming for 20-80% confluence. Cells were detached from tissue culture flasks by rinsing with D-PBS followed by incubation with D-PBS and 2 mM EDTA at 37° C. until detached.

### (f) SW.3G1 Did not Recognize Target Cells by Known Mechanisms

The Vγ9Vδ2 T-cell activator (E)-4-Hydroxy-3-methyl-but-2-enyl pyrophosphate (HMB-PP) (Sigma Aldrich) was reconstituted in DMSO and added directly to assay wells. The following monoclonal Abs were used for blocking assays: anti-HLA, -B, -C(clone W6/32, Biolegend), anti-HLA-DR, -DP, -DQ (clone Tu39, Bioloegend), anti-EPCR (polyclonal, R&D systems), anti-MICA/MICB (clone 6D4, BioLegend) and anti-CD1d (clone 51.1, Miltenyi Biotech) were used at a final concentration of 10 μg/mL.

### (g) Gene Trapping by Whole Genome CRISPR

A whole genome CRISPR/Cas9 library approach was used (FIG. 5 for an overview). Whole genome targeted LCL-9909 and KBM7s using the GeCKO v2 sub-libraries A and B (Adgene plasmid, #1000000048, kindly provided by Dr. Feng Zhang (Patel et al., 2017)) were used for selection by SW.3G1. Briefly, successfully transduced target cells selected with puromycin were co-incubated with SW.3G1 at a predefined ratio for 2-3 weeks in 96 U well plates. Activation assays (as above) were performed with pre- and post-selected target cells to confirm loss of SW.3G1 activity towards the selected cells. Genomic DNA from the target cells that had survived two rounds of selection with SW.3G1 was used for next generation sequencing to reveal inserted guide RNAs and candidate genes.

### (h) Confirming SCNN1A Role in Target Cell Recognition

Lentiviral particles were generated by calcium chloride transfection of HEK 293T cells and concentrated by ultracentrifugation prior to transduction of target cells using 8 μg/mL of polybrene and spinfection. gRNAs were cloned into the pLentiCRISPR v2 plasmid (kindly provided by Dr. Feng Zhang, Addgene plasmid 52961), which encodes the SpCas9 protein and a puromycin resistance marker gene (pac, puromycin N-acetyltransferase), and co-transfected with packaging and envelope plasm ids pMD2.G and psPAX2 (all from Addgene). Full-length codon optimized SCNN1A transgene (Isoform 1, UniProt P37088-1) was cloned in to a 3^{rd} generation lentiviral transfer vector pELNS (kindly provided by Dr. James Riley, University of Pennsylvania, PA). The pELNS vector contains rat CD2 (rCD2) gene for selection of cells using an anti-rCD2 PE Ab (OX-34, BioLegend). SCNN1A expression in target cells was assessed using the rabbit anti-SCNN1A polyclonal antibody (PA1-902A, ThermoFisher Scientific) for flow cytometry (data not sown) and western blot analysis according to the manufacturer's instructions.

(i) Transduction of Polyclonal T-Cells with the SW.3G1 TCR Confers Target Cell Recognition

Codon optimized, full length TCR chains, separated by a self-cleaving 2A sequence, were synthesized (Genewiz) (SEQ ID NO: 23; corresponding protein sequence SEQ ID NO: 24) and cloned into the 3^{rd} generation lentiviral transfer vector pELNS (kindly provided by Dr. James Riley, University of Pennsylvania, PA). The pELNS vector contains a rat CD2 (rCD2) marker gene separated from the TCR by another self-cleaving 2A sequence. Additionally, cells were co-transduced with a gRNA to ablate TCRβ chain expression in recipient cells by targeting both TCR-β constant domains (manuscript currently at Blood for publication). Lentiviral particles were generated by calcium chloride transfection of HEK293T cells. TCR transfer vectors were co-transfected with packaging and envelope plasmids pMD2.G, pRSV-Rev and pMDLg/pRRE. Lentiviral particles were concentrated by ultracentrifugation prior to transduction of CD8+ T-cells using 5 μg/ml of polybrene, with the CD8+ T-cells purified by magnetic separation (Miltenyi Biotec) from three healthy donors 24 h in advance and activated overnight with CD3/CD28 beads (Dynabeads, Life Technologies) at 3:1 bead:T-cell ratio. T-cells that had taken up the virus were selected by incubation with 2 μg/ml puromycin (TCRβ chain knock-out) and enriched with anti-rCD2 PE Ab (OX-34, BioLegend) followed by anti-PE magnetic beads (Miltenyi Biotec). 14 d post transduction T-cells were expanded with allogeneic feeders and PHA. TCR transduced cells were used in longterm killing assays whereby LCL.174 targets were plated in duplicate at the density of 50,000 cells/well in 96 U well plates. SW.3G1 was added to the target and incubated for 7 days. Target cells were also plated without T-cells, to serve as a 100% survival control. Cells were harvested, washed with PBS, and stained with Vivid and anti-CD3 antibody (to exclude T-cells). As an internal control, CountBright™ Absolute Counting Beads (Life Technologies) were added to each well prior to harvesting/washing (approximately 10,000 beads/well). The samples were the acquired on FACS Canto II, and at least 1,000 bead events were acquired per sample. The survival of target cells was calculated according to the following formula:

$$\% survival = \frac{\begin{matrix}number of experimental cell events / \\ number of experimental bead events\end{matrix}}{number of control cell events / number of control bead events} \times 100 \%$$

### (j) Results

### Clone Characterisation

1. Purified γδ T-cells from a healthy donor (9909) primed and re-stimulated with a pool of three non-autologous lymphoblastoid cell lines (-0439, -pt146 and -HOM-2). Reactivity towards each of the cells lines was tested at day 28 (FIG. 1). The T-cell line also recognized autologous LCL-9909 (FIG. 1).

2. T-cells from the aforementioned line were flow cytometry sorted based on reactivity to each of the LCLs and their TCRs analysed by next generation sequencing (FIG. 1). For the γ-chain sequencing, two unique CDR3s were present with variable chains TRGV9 and TRGV3. For the δ-chains, three CDR3s were present with variable chains TRDV1 and TRDV2.

3. T-cells clones procured from the donor 9909 T-cell line expressed a γ3δ1 TCR and CDR3s CATWDRRDYKKLF and CALGVLPTVTGGGLIF for each respective chain (FIG. 1 and FIG. 2). All the clones that grew expressed the same TCR. This clone was named SW.3G1.

Ab staining of SW.3G1 confirmed expression of the Vδ1 chain, and αβ TCR−/CD8 low/CD4− (FIG. 1B).

4. Activation assays using TNFα and CD107a as the readouts confirmed SW.3G1 reactivity towards autologous LCL-9909 and non-autologous LCL-0439 (FIG. 3A). Donors 9909 and 0439 are completely HLA mismatched for both MHC class I and class II alleles, therefore SW.3G1 is recognizing target cells in an HLA-independent manner. SW.3G1 was also able to lyse LCL-9909 and -0439 and is therefore cytotoxic (FIG. 3B). The recognition of the LCLs was dependent on the immortalization process when EBV infects a B-cell, as autologous healthy B-cells purified directly ex-vivo from 9909 did not act as targets for SW.3G1 (FIG. 3C). Similarly, the healthy cells CIL-1 (epithelial cell) and Hep2 (hepatocyte) did not elicit SW.3G1 activation (FIG. 3D).

5. SW.3G1 was able to lyse LCLs from all 24 donors tested (FIG. 3B for the autologous LCL and FIG. 4A for 23 non-autologous LCLs) providing further confirmation that SW.3G1 is acting in a HLA independent manner. Furthermore, LCL 0.174 (FIG. 4A, 4th bar from the left) only expresses one copy of chromosome 6, the human chromosome that carries the MHC locus. The chromosome 6 in cell LCL 0.174 contains a large deletion and does not carry genes for MHC class II and many components involved in MHC class I antigen processing.

6. SW.3G1 killed 23 cancer cell lines that originate from 8 different tissues: skin/melanoma, kidney, colon, breast, blood/leukemia, lung, cervix and bone. (FIG. 4B).

7. SW.3G1 did not respond to the known γδ T-cell antigen, HMB-PP (FIG. 5A), which leads to recognition of pathogen infected cells by the Vγ9Vδ2 TCR T-cell subset in a similar manner to recognition of the self pyrophosphate. This pathway requires target cells to express Butyrophillin 3A1. SW.3G1 reactivity towards LCL-9909 (autologous) -0439 and -pt146 was not hindered by inclusion of blocking antibodies that bind known γδ T-cell ligands: Major Histocompatibility Complex (MHC) Class-I related chain A and B (MICA/MICB), EPCR Endothelial Protein C Receptor (EPCR) and CD1d (FIG. 5B). MHC class I and class II also failed to block SW.3G1 activation. Although not an extensive exclusion process, these data suggested that SW.3G1 might recognize an unknown γδ TCR ligand at the surface of cancer cells. Therefore, a whole genome CRISPR/Cas9 library approach was adopted to find candidate genes/proteins involved in SW.3G1 recognition of target cells (FIG. 6).

8. Whole genome CRISPR/Cas9 libraries were used to create gene knockouts in autologous LCL-9909 and the haploid myeloid leukaemia cell line KBM7. Both libraries were co-incubated with SW.3G1 for successive rounds of selection to enrich for target cells containing gRNAs that allowed escape from SW.3G1-mediated lysis (FIG. 7A). SW.3G1 reactivity dropped from 59% for pre-selected LCL-9909 to 12% post-selection. For KMB7s reactivity went from 12% to 4.2%. The post-selected LCL-9909s and KBM7s were used for next generation sequencing to identify gRNAs that had been enriched. Key genes were identified with 4 of the total 7 genes shared between the LCL-9909 and KBM7 libraries (FIGS. 7B and 7C). Guides specific for the gene SCNN1A (also used here to describe the encoded protein), which encodes for the protein Sodium Channel Epithelial 1 Alpha Subunit, were highly enriched present in both libraries (FIGS. 7B&C). SCNN1A gene and protein aliases are shown in FIG. 8. The protein is cell surface expressed and therefore a good candidate for further exploration. SCNN1A has 6 splice variant isoforms and various naturally occurring mutations (FIGS. 8 and 9).

9. LCL.174 transduced with SCNN1A gRNA from the whole genome library (GeCKO, gRNA-1, SEQ ID NO: 12) or a different guide designed in-house (gRNA-2, SEQ ID NO: 13) (FIG. 10A) were no longer targets of SW.3G1, thereby confirming SCNN1A's role in target cell recognition (FIG. 10B), with lysis falling to below 5% for the knockout cell lines compared to 100% killing for the wildtype cells. SCNN1A gene knockout lines were created in two cancer cells, which either partially or completely escaped lysis by SW.3G1 (FIG. 10C). It is noteworthy that the SCNN1A knockout cells created throughout this study were used as lines, and not cloned before performing assays. This may account for the residual reactivity seen for some of the ‘knockout’ cell lines as a minority proportion of the cells within a knockout line probably still express SCNN1A, due to escape from puromycin selection and/or unsuccessful ablation of the SCNN1A gene. Western blot analysis of the SCNN1A knockout MDA-MB-231 cells used for SW.3G1 activation assays revealed a substantial reduction of the SCNN1A protein in the knockout cell line compared to the wildtype cells (FIG. 10C) confirming the gene knockout. The Ab used can recognise all SCNN1A isoforms (FIG. 9). We also noted that the SCNN1A knockout cells became less viable with extensive culture (3+ weeks) and in some cases cell division halted completely. This observation was unique to the SCNN1A gRNA as the same cell lines transduced with gRNAs for many other genes did not exhibit the same change in cell growth and vitality. This result suggested that the SCNN1A gene is essential for long-term growth of cells in culture.

10. Transfer of the SW.3G1 TCR in to polyclonal CD8+ T-cells from three healthy donors conferred reactivity to target cell LCL-pt146 (FIG. 11A). TCR transduced cells exhibited the same functional profile to SCNN1A knockout cells as described above for the SW.3G1 clone (FIG. 11B). To compliment the SCNN1A knockout data, and to further confirm the role of SCNN1A in target cell recognition, we transduced the knockout cells with the SCNN1A gene. The introduction of a native SCNN1A gene to knockout cells expressing the SCNN1A gRNAs would lead to gene ablation of the transgene. Therefore, a codon-optimized gene was introduced, different to the DNA sequence of the native gene (Isoform 1 UniProt P37088-1, FIG. 9), but expressing the same protein. Killing of the gRNA-1 or gRNA-2 transduced cells was ablated but could be restored by expressing the SCNN1A gene in the knockout cells (FIG. 11B).

### (k) Conclusion

The SW.3G1 TCR enables T-cells to recognise a wide range of tumours. Recognition occurs via the SCNN1A gene product. SW.3G1 T-cell clone recognises a cancer-cell specific SCNN1A ligand in the absence of MHC restriction.

This invention centres around the TCR identified in T-cell clone SW.3G1. This TCR recognises a wide range of cancer cells through the expression of SCNN1A. This TCR does not recognise non-tumour cells. CRISPR/Cas9 knockout of SCNN1A from tumour lines or antibody blocking confirmed there TCR requires the SCNN1A gene product for recognition of tumour cells. The SW.3G1 TCR can be used in a variety of different cancer immunotherapy strategies. The broad tumour recognition and human leukocyte antigen (HLA)-independence of recognition unlocks exciting possibilities for pan-cancer, pan-population immunotherapies using this TCR.

### Example 2

Design of a mutant TCR

### Example 2a

A mutant version of the SW.3G1 TCR was designed and prepared in which a R to N mutation was introduced into the constant region within the extracellular region of the δ chain. This mutation was made in order to improve stability of the domain. See SEQ ID NOs: 7 and 8.

### Example 3

### Preparation of an Artificial T-Cell Expressing the TCR of the Invention

Expression of the T cell receptor of the invention (“SW.3G1 T cell receptor”) in patients' T cells is a potential therapeutic approach for cancer immunotherapy. DNA encoding the γ and δ chains of the T cell receptor, separated with a cleavable read-through linker, such as those of the 2A family can be expressed in a lentiviral vector (see Example 1(i)) This vector when transduced into patients' CD8 T cells will drive expression of the SW.3G1 T cell receptor γ and δ chains at a 1:1 stoichiometry and thus allow a mature functional SW.3G1 TCR to be expressed on the cell surface.

To produce CD8 T cells expressing a SW.3G1 recombinant receptor the following procedures are followed. Firstly, the DNA sequences of the TCR (such as the mutant of Example 2) are synthesised from assembled oligonucleotides and cloned into a lentiviral vector. Features of the lentiviral system to be utilised include a lentiviral vector incorporating self-inactivating long terminal repeats (LTR). The viral vector encodes the entire coding sequence, including the signal sequences, of the TCR γ and δ chains separated by a read-through self-cleaving linker of the 2A family. The transcription of the mRNA encoding the TCR chains is driven by a constitutive promoter such as the cytomegalovirus (CMV) promoter or elongation factor 1a (EF1a) promoter.

The SW.3G1 encoding lentiviral plasmid vector is packaged into a virus using three additional packaging plasmids. The packaging plasmids encode 1) gag-pol 2) rev and 3) the envelope protein of vesicular stomatitis virus (VSV-G). Separation of the virus genome into 4 plasmids increases the safety of the virus and minimises the chances of recombination events leading to replication competent lentivirus (RCL) formation. Using cationic lipid transfection reagents, the vector and three packaging plasmids are simultaneously transfected into the packaging cell line HEK293T, in which replication-incompetent, SW.3G1 TCR-encoding lentivirus is assembled and secreted into the packaging cell culture supernatant. HEK293T cells may be grown attached to plastic or adapted for growth in suspension. Virus particles are concentrated from the cell supernatant by high speed centrifugation, and in some cases may be filtered through 0.45 μm pore filters to remove cellular debris and cryopreserved prior to transduction of CD8 T cells.

To purify patient peripheral blood CD8 T cells, apheresis is first performed on patients to isolate large numbers of peripheral blood mononuclear cells (PMBC) typically in the range of 10-20 billion mononuclear cells per patient. CD8 T cells can isolated from these PBMCs using magnetic bead isolation. Microscopic paramagnetic beads coated with antibodies against CD8 are incubated with the PBMC, followed by separation on columns within a strong magnetic field. CD8-negative cells are eluted from the column by washing with serum albumin-containing phosphate buffered saline (SA/PBS). Following washing, the CD8 bead-labelled cells are released by removal from the magnet and washing with SA/PBS. The CD8 T cells are then enumerated and incubated in cell culture medium in advance of activation and transduction with lentivirus encoding the TCR of the invention.

Transduction of CD8 T cells with TCR-encoding lentiviral particles is achieved by incubating CD8 T cells in cell culture media into which lentiviral particles are added. CD8 cells may be in the resting state or pre-activated with antibodies against CD3 and CD28 with addition of cytokines including interleukin-2 and interleukin-7. Following 24-48 hours after transduction the cells are expanded for infusion back into patients. Expansion is performed with antibodies against CD3 and CD28, either in soluble or bead-bound form with addition of interleukin-2 for 14 days. In addition to interleukin-2, other cytokines such as interleukin-7 may also be added to the cultures. Expansion may also be performed using autologous feeder PBMCs which are irradiated with 30 Gray gamma irradiation to inhibit proliferation plus anti-CD3 antibody plus interleukin-2 at 3000-6000 IU/ml for 14 days.

### Example 4

### Applications of a Soluble Form of the TCR of the Invention

Solubilisation of TCRs opens up the ability to utilise TCRs in a number of applications, to include but not limited to, precise affinity measurements of the TCR for its ligand, crystallography-based structure solving, multimerization, and the generation of clinical therapies utilising the TCR as a targeting mechanism.

### (a) Generation of Soluble, Monomeric SW.3G1 TCR for Further Application

Expression of a soluble SW.3G1 TCR (“SW.3G1 sTCR”), or a soluble mutant form as described in Example 2, could be used to characterise binding kinetics to its cognate SCNNA1 gene ligand. Briefly, SW.3G1 sTCR can be tested by surface plasmon resonance (SPR), biolayer interferometry (BLI), or similar, to determine biophysical kinetics of the interaction between the soluble TCR and a derivative of its cognate SCNNA1 gene ligand. For example, in the case of SPR, the its cognate SCNNA1 gene ligand is immobilised on specialised biosensor chips and concentrated SW.3G1 sTCR is passed over the chip and the interaction measured by SPR. From this, the affinity and binding half-life can be determined by calculation of the on- (kₐ or kₒₙ) and off-rates (k_{d} or k_{off}).

(b) Generation of Multimeric SW.3G1 sTCRs for Further Application

The DNA sequence for SW.3G1 sTCR can also be modified for example to contain an acceptor tag peptide for biotin at the C-terminus of one of the chains. Following expression of such a construct, this acceptor tag can specifically be labelled with biotin via an enzymatic reaction using a biotin ligase such as BirA. Such a method will create a uniform biotinylation of the SW.3G1 sTCR which will allow it to be multimerised via binding to streptavidin, for generation of tetramers, pentamers or dextramers. These multimers can be further modified via the addition of a molecule such as phycoerythrin (PE), a fluorophore that allows detection of the multimer via fluorescence. The SW.3G1 sTCR multimeric complex could then be used to identify SCNNA1 gene ligand expressing cells via incubation of the molecule with target cells, and detection and/or sorting via methods such as fluorescent activated cell sorting (FACS).

(c) Generation of modified SW.3G1 sTCRs for potential therapeutic application

The SW.3G1 sTCR binding to its cognate ligand in vivo is unlikely to confer any therapeutic benefit. In order to generate a therapeutic agent with clinical effect, further modifications of the sTCR are required. The SW.3G1 TCR recognises its target on a number of different cancer cells and cancer cell-derived cell lines. Therefore, to generate a reagent with therapeutic utility in the oncology setting, a SW.3G1 sTCR would need to have a function introduced that would induce cell death of the target cell, either directly or indirectly.

To modify SW.3G1 sTCR for this purpose, an effector function could be introduced, such as an anti-CD3 agonistic antibody, to engage and redirect T cell activity. An anti-CD3 antibody optionally in scFv format could be added to the N or C terminus of either the γ or δ chain of the SW.3G1 sTCR, via a short, non-immunogenic linker sequence (such as GGGGS, SEQ ID NO: 25). Such a fusion protein would have bispecific properties.

### REFERENCES

Chen, J., T R Kleyman and S. Sheng. 2014 Deletion of α-subunit exon 11 of the epithelial Na+ channel reveals a regulatory module. Am J Physiol. Renal Physiol 306, F561-7.
Donia, M., Kjeldsen, J. W., Andersen, R., Westergaard, M. C. W., Bianchi, V., Legut, M., Attaf, M., Szomolay, B., Ott, S., Dolton, G., Lyngaa, R., Hadrup, S. R., Sewell, A. K., Svane, I. M., 2017. PD-1 polyfunctional T cells dominate the periphery after tumor-infiltrating lymphocyte therapy for cancer. Clin. Cancer Res. clincanres.1692.2016.
Ekeruche-Makinde, J., Clement, M., Cole, D. K., Edwards, E. S. J., Ladell, K., Miles, J. J., Matthews, K. K., Fuller, A., Lloyd, K. A., Madura, F., Dolton, G. M., Pentier, J., Lissina, A., Gostick, E., Baxter, T. K., Baker, B. M., Rizkallah, P. J., Price, D. A., Wooldridge, L., Sewell, A. K., 2012. T-cell receptor-optimized peptide skewing of the T-cell repertoire can enhance antigen targeting. J. Biol. Chem. 287, 37269-81.
Haney, D., Quigley, M. F., Asher, T. E., Ambrozak, D. R., Gostick, E., Price, D. A., Douek, D. C., Betts, M. R., 2011. Isolation of viable antigen-specific CD8+ T cells based on membrane-bound tumor necrosis factor (TNF)-alpha expression. J. Immunol. Methods 369, 33-41.
Legut, M. G Dolton, A. A. Mian, O. Ottmann and A. K. Sewell. 2017 CRISPR-mediated TCR replacement generates superior anticancer transgenic T-cells. Blood [Epub ahead of print] doi: https://doi.org/10.1182/blood-2017-05-787598
Patel, S. J., Sanjana, N. E., Kishton, R. J., Eidizadeh, A., Vodnala, S. K., Cam, M., Gartner, J. J., Jia, L., Steinberg, S. M., Yamamoto, T. N., Merchant, A. S., Mehta, G. U., Chichura, A., Shalem, O., Tran, E., Eil, R., Sukumar, M., Guijarro, E. P., Day, C.-P., Robbins, P., Feldman, S., Merlino, G., Zhang, F., Restifo, N. P., 2017. Identification of essential genes for cancer immunotherapy. Nature 548, 537-542.
Theaker, S. M., Rius, C., Greenshields-Watson, A., Lloyd, A., Trimby, A., Fuller, A., Miles, J. J., Cole, D. K., Peakman, M., Sewell, A. K., Dolton, G., 2016. T-cell libraries allow simple parallel generation of multiple peptide-specific human T-cell clones. J. Immunol. Methods 430, 43-50.

1. A T cell receptor (TCR) or a binding fragment of a TCR which binds a tumour antigen comprising:
a) a γ chain that comprises a CDR3 (CDR3γ) comprising an amino acid sequence that is at least 88% identical to the amino acid sequence of SEQ ID NO: 1; and/or
b) a δ chain that comprises a CDR3 (CDR3δ) comprising an amino acid sequence that is at least 88% identical to the amino acid sequence of CALGVLPTVTGGGLIF (SEQ ID NO: 2).

2-5. (canceled)

6. The TCR or binding fragment of claim 1, wherein:
a) the γ chain comprises CDR1γ, CDR2γ, and CDR3γ amino acid sequences that are at least 88% identical to the amino acid sequences of SEQ ID NOs: 3, 4, and 1, respectively; and/or
b) the δ chain comprises CDR1δ, CDR2δ, and CDR3δ amino acid sequences that are at least 88% identical to the amino acid sequences of SEQ ID NOs: 5, 6, and 2, respectively.

7. (canceled)

8. (canceled)

9. The TCR or binding fragment of claim 1 wherein:
a) the γ chain comprises an extracellular region comprising an amino acid sequence that is at least 88% identical to the amino acid sequence of SEQ ID NO: 7 or 17; and/or
b) the δ chain comprises an extracellular region comprising an amino acid sequence that is at least 88% identical to the amino acid sequence of SEQ ID NO: 8, 14, or 18.

10-12. (canceled)

13. The TCR or binding fragment of claim 1, wherein:
a) the amino acid sequence of the TCR or binding fragment is artificial;
b) at least one amino acid is substituted, added or deleted relative to the wildtype sequence;
c) at least one amino acid is substituted, added or deleted in a framework region, a CDR or a constant region relative to the wildtype sequence;
d) at least one amino acid is substituted, added or deleted relative to the wildtype sequence, wherein the at least one amino acid is not located in any CDR;
e) the TCR is of soluble form;
f) the TCR or tumour-specific binding fragment is specific for at least one SCNNA1 gene product isoform;
g) the TCR or tumour-specific binding fragment of a TCR binds to at least one of the SCNNA1 gene product isoforms encoded by the amino acid sequence of SEQ ID NOs: 29 to 34; and/or
h) the TCR or tumour-specific binding fragment of a TCR binds to the extracellular domain of at least one SCNNA1 gene product isoform.

14-20. (canceled)

21. The TCR or binding fragment of claim 1, wherein:
a) the γ chain comprises an amino acid sequence that is at least 88% identical to amino acids 19-307 of SEQ ID NO: 15;
b) the δ chain comprises an amino acid sequence that is at least 88% identical to amino acids 21-290 of SEQ ID NO: 16.

22. The TCR or binding fragment of claim 1, wherein the γ chain and the δ chain each comprise a constant region in which the constant region is replaced by the corresponding sequence of the constant region of a murine TCR or a variant thereof.

23. A polynucleotide encoding the TCR or binding fragment of claim 1, optionally wherein the polynucleotide is comprised within a vector, optionally a viral vector.

24-27. (canceled)

28. A cell comprising the polynucleotide of claim 23, optionally wherein the cell is a T cell.

29-31. (canceled)

32. An ex vivo process comprising (i) obtaining T-cells from a patient, (ii) optionally expanding the T-cells (iii) transforming the T-cells with the vector of claim 25; and (iv) reintroducing said transduced T-cells into the patient.

33. A method of treating cancer in a subject in need thereof comprising administering to the subject the cell of claim 28.

34. A pharmaceutical composition comprising the cell of claim 28 and a pharmaceutically acceptable carrier.

35. (canceled)

36. A bispecific construct comprising the TCR or binding fragment of claim 1 and an immune cell activating component or ligand that binds to and activates an immune cell, optionally wherein the immune cell activating component or ligand that binds to and activates an immune cell binds to CD3.

37. (canceled)

38. A fusion protein comprising the TCR or binding fragment of claim 1 and a heterologous protein.

39-49. (canceled)

50. A polynucleotide encoding the bispecific construct of claim 36, optionally wherein the polynucleotide is comprised within a vector, optionally wherein the vector is a viral vector.

51. (canceled)

52. (canceled)

53. A cell comprising the polynucleotide of claim 50, optionally wherein the cell is a T cell.

54. (canceled)

55. (canceled)

56. A method of treating cancer in a subject in need thereof, comprising administering to the subject a therapeutically effective amount of the cell of claim 53.

57. (canceled)

58. A polynucleotide encoding the fusion protein of claim 38, optionally wherein the polynucleotide is comprised within a vector, optionally wherein the vector is a viral vector.

59. A cell comprising the polynucleotide of claim 58, optionally wherein the cell is a T cell.

60. A method of treating cancer in a subject in need thereof comprising administering to the subject a therapeutically effective amount of the cell of claim 59.

61. A method of producing a TCR, the method comprising:
a) introducing into a host cell the polynucleotide of claim 23; and
b) culturing the host cell so that the polynucleotide is expressed and the TCR is produced.

62. A method of producing a bispecific construct, the method comprising:
a) introducing into a host cell the polynucleotide of claim 50; and
b) culturing the host cell so that the polynucleotide is expressed and the bispecific construct is produced.

63. A method of producing a fusion protein, the method comprising:
a) introducing into a host cell the polynucleotide of claim 58; and
b) culturing the host cell so that the polynucleotide is expressed and the bispecific construct is produced.
